(19) United States
(12) Patent Application Publication
(10) Pub. No.: US 2026/0086085 A1
(43) Pub. Date: Mar. 26, 2026

(54) METHODS OF ANALYZING LIPID NANOPARTICLES IN PHYSIOLOGICAL FLUIDS

(71) Applicant: AGILENT TECHNOLOGIES, INC., Santa Clara, CA (US)

(72) Inventors: Brian Liau, Singapore (SG); Ravindra Gudihal, Singapore (SG)

(21) Appl. No.: 19/113,575

(22) PCT Filed: Oct. 16, 2023
(86) PCT No.: PCT/US2023/076951

§ 371 Date: Mar. 20, 2025

Related U.S. Application Data
(60) Provisional application No. 63/379,852, filed on Oct. 17, 2022.

(51) Int. Cl. G01N 33/53; G01N 30/14; G01N 30/74; G01N 30/88; G01N 33/92

(52) U.S. Cl. CPC G01N 33/5308; G01N 30/14; G01N 30/74; G01N 30/88; G01N 33/92; G01N 2030/8813

(57) ABSTRACT

Methods and systems for analyzing lipid nanoparticles using size exclusion chromatograph coupled with multi angle light scattering are disclosed.

### CROSS-REFERENCE TO RELATED APPLICATIONS

This application claims the benefit of U.S. Provisional Application No. 63/379,852, filed Oct. 17, 2022. The contents of the aforementioned application are hereby incorporated by reference in its entirety.

### BACKGROUND

As one of the breakthrough technologies to emerge from the COVID-19 pandemic, mRNA-loaded lipid nanoparticles (mRNA-LNPs) have proven to be a safe and efficacious means of expressing proteins in human cells and tissues without mutagenic risk. LNPs have also been used for clinical delivery of siRNA and are now being investigated for many therapeutic applications beyond vaccination. This includes cancer immunotherapy and rare genetic diseases. Copious resources have been allocated to expanding production capacity for approved lipid excipients, and to discovering new LNP formulations with improved properties. Thus, there exists a need to develop new methods and systems that quantitatively analyze lipid nanoparticles in the presence of physiological fluids.

### SUMMARY

In an aspect, the disclosure provides a method of analyzing a lipid nanoparticle (LNP). The method comprises: (optional) acquiring a sample comprising the LNP (e.g., in a physiological fluid); subjecting the sample to a size-exclusion chromatography (SEC); and acquiring a multi angle light scattering (MALS) signal from the sample, thereby analyzing the LNP.

In some embodiments, the method further comprises: subjecting a reference sample to the SEC; and acquiring an MALS signal from the reference sample. In some embodiments, the method further comprises comparing the MALS signal from the sample to the MALS signal from the reference sample.

In some embodiments, the method further comprises subjecting the sample to LC-MS/MS, e.g., to determine (e.g., quantify) the component(s) of the LNP. In some embodiments, the method further comprises acquiring the UV absorbance (e.g., at λ=260 nm) for the sample, e.g., to determine (e.g., quantify) the amount of nucleic acid in the LNP.

In some embodiments, the method determines the stability of the LNP in the sample. In some embodiments, the method determines the purity of the LNP in the sample. In some embodiments, the method is suitable for monitoring manufacturing of the LNP. In some embodiments, the method is suitable for determining the pharmacokinetics (PK) of the LNP.

In some embodiments, the sample is not acquired from a subject, e.g., a physiological fluid reconstituted with the LNP. In some embodiments, the sample is acquired from a subject. In some embodiments, the subject is a healthy subject. In some embodiments, the subject has, or is likely to have, a disorder, e.g., an infection, a cancer, or an autoimmune disorder. In some embodiments, the subject is a human. In some embodiments, the subject is an animal, e.g., a mammal, e.g., a mouse, a rat, or a primate.

In some embodiments, the SEC is performed in a single-column configuration. In some embodiments, the SEC is performed in a dual-column configuration, e.g., using two columns with different pore sizes, e.g., a first column with a first average pore size and a second column with a second average pore size. In some embodiments, the first average pore size is greater than the second average pore size. In some embodiments, the first average pore size has a molecular weight cut-off (MWCO) of about 1 MDa or more, e.g., about 2 MDa or more, 5 MDa or more, 10 MDa or more, 15 MDa or more, or 20 MDa or more. In some embodiments, the first average pore size has an MWCO of about 20 MDa or less, e.g., about 15 MDa or less, 10 MDa or less, 5 MDa or less, 2 MDa or less, or 1 MDa or less. In some embodiments, the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa. In some embodiments, the second average pore size has an MWCO of about 10 MDa or less, e.g., about 5 MDa or less, 2 MDa or less, 1 MDa or less, 0.5 MDa or less, 0.2 MDa or less, or 0.1 MDa or less. In some embodiments, the second average pore size has an MWCO of about 0.1 MDa or more, e.g., about 0.2 MDa or more, 0.5 MDa or more, 1 MDa or more, 2 MDa or more, 5 MDa or more, or 10 MDa or more. In some embodiments, the second average pore size has an MWCO of about 0.1 MDa to 10 MDa, e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.

In some embodiments, the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 250 mm.

In some embodiments, the SEC is performed using a polymer-based column. In some embodiments, the SEC is not performed using a silica-based column.

In some embodiments, the SEC is performed under an aqueous condition, e.g., with a bio-compatible buffer system, e.g., a phosphate buffer, a bicarbonate buffer, an acetate buffer, a Tris buffer, or any of the Good's buffers (e.g., as described in Good et al. Biochemistry. 1966; 5(2):467-77; Good and Izawa Methods Enzymol. 1972:24:53-68; Ferguson et al. Anal Biochem. 1980 May; 104(2):300-10), e.g., any of MES. Bis-tris methane, ADA, Bis-tris propane, PIPES ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS, or a combination thereof. In some embodiments, the SEC is not performed sing an organic solvent (e.g., THF) as a mobile phase.

In some embodiments, the MALS is performed at a scattering angle of 15° and 90°. In some embodiments, the MALS is performed using a laser wavelength of about 500 nm to 800 nm, e.g., about 600 nm to 700 nm, 500 nm to 700 nm, 600 nm to 800 nm, 500 nm to 600 nm, 700 nm to 800 nm, e.g., about 658 nm. In some embodiments, the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL. In some embodiments, the MALS is performed using a scattering volume of about 0.001 μL to 0.1 μL, e.g., about 0.005 μL to 0.05 μL, 0.001 μL to 0.05 μL. 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL. In some embodiments, the MALS is performed at a temperature range of about 20° C. to 70° C., e.g., about 25° C. to 75° C. or 30° C. to 60° C. In some embodiments, the MALS is performed with a temperature stability of no more than ±1° C., e.g., no more than ±0.5° C. or ±0.2° C. In some embodiments, the MALS is performed at a pH range of about 1-12, e.g., about 2-11 or 2-10.

In some embodiments, the size of the LNP is determined, e.g., the hydrodynamic radius (Rₕ) of the LNP is determined. In some embodiments, the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined. In some embodiments, the polydispersity index is determined, e.g., by calculating the ratio of M_{w} to Mₙ. In some embodiments, the R_{gw} of the LNP is determined, e.g., using a Zimm or partial Zimm approach (e.g., as described in Wyatt. Analytica Chimica Acta. 1993. 272: 1-40). In some embodiments, the half-life the LNP is determined. In some embodiments, the stability of the LNP is determined in accordance with a method described herein, e.g., a method described in Example 1.

In some embodiments, the method comprises comparing the acquired MALS signals directly. In some embodiments, the method comprises comparing the acquired MALS signals indirectly.

In some embodiments, a plurality of LNPs in the sample is analyzed, e.g., in accordance with a method described herein.

In some embodiments, the method does not comprise a step of recovering the LNP, e.g., by ultracentrifugation. In some embodiments, the method does not comprise a step of diluting the sample, e.g., to remove a high molecular weight component (e.g., plasma or serum component) that interferes with detection (e.g., detection by DLS). In some embodiments, the method does not comprises a step of labeling the LNP, e.g., with a fluorophore. In some embodiments, the method quantitatively detects the LNP in the sample.

In some embodiments, the LNP is loaded with a nucleic acid. In some embodiments, the LNP is not loaded with a nucleic acid. In some embodiments, the nucleic acid is a therapeutic nucleic acid (TNA). In some embodiments, the nucleic acid is an mRNA. In some embodiments, the nucleic acid is a vaccine. In some embodiments, the nucleic acid is a non-coding RNA, e.g., a small non-coding RNA. In some embodiments, the nucleic acid is a small interfering RNA (siRNA), an antisense oligonucleotide (ASO), or a microRNA (miRNA). In some embodiments, the nucleic acid is a guide RNA (gRNA). In some embodiments, the nucleic acid is a DNA. In some embodiments, the nucleic acid is an aptamer. In some embodiments, the nucleic acid comprises one or more modified nucleotides. In some embodiments, the nucleic acid is single stranded. In some embodiments, the nucleic acid is double stranded.

In some embodiments, the sample comprises a physiological fluid. In some embodiments, the physiological fluid is plasma. In some embodiments, the physiological fluid is serum. In some embodiments, the physiological fluid is blood. In some embodiments, the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9. In some embodiments, the physiological fluid has a pH of serum or plasma (e.g., 7.3-7.5). In some embodiments, the physiological fluid has a pH of a tumor microenvironment (e.g., pH 5.6 to 6.8).

In another aspect, the disclosure provides a method of determining the stability of a lipid nanoparticle (LNP) in a subject. The method comprises: (optionally) acquiring a first sample comprising the LNP from the subject; subjecting the first sample to a size-exclusion chromatography (SEC); acquiring a multi angle light scattering (MALS) signal from the first sample: (optionally) acquiring a second sample comprising the LNP from the subject; subjecting the second sample to the SEC; acquiring a second MALS signal from the second sample; and comparing the first MALS signal and the second MALS signal, wherein the comparison between the first MALS signal and the second MALS signal is indicative of the stability of the LNP in the subject, thereby determining the stability of the LNP in the subject.

In some embodiments, the subject has been administered with the LNP, e.g., at least 1, 6, 12, 18, or 24 hours, or 1, 2, 3, 4, 5, 6, or 7 days, before the first sample comprising the LNP is acquired. In some embodiments, the second sample is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first sample is acquired.

In some embodiments, the method further comprises: (optionally) acquiring a third sample comprising the LNP from the subject; subjecting the third sample to the SEC; acquiring a third MALS signal from the third sample; and comparing the third MALS signal with the first MALS signal, the second MALS signal, or both, wherein the comparison is indicative of the stability of the LNP in the subject. In some embodiments, the third sample is acquired after the second sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second sample is acquired.

In some embodiments, the method further comprises: (optionally) acquiring a fourth sample comprising the LNP from the subject: subjecting the fourth sample to the SEC; acquiring a fourth MALS signal from the fourth sample; and comparing the fourth MALS signal with one or more (e.g., all) of the first MALS signal, the second MALS signal, or the third MALS signal, wherein the comparison is indicative of the stability of the LNP in the subject. In some embodiments, the fourth sample is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third sample is acquired.

In some embodiments, the method further comprises: (optionally) acquiring a fifth sample comprising the LNP from the subject; subjecting the fifth sample to the SEC; acquiring a fifth MALS signal from the fifth sample; and comparing the fourth MALS signal with one or more (e.g., all) of the first MALS signal, the second MALS signal, the third MALS signal, or the fourth MALS signal, wherein the comparison is indicative of the stability of the LNP in the subject. In some embodiments, the fifth sample is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth sample is acquired.

In some embodiments, the LNP in the second or subsequent sample has a change to the LNP composition, e.g., lipid, nucleic acid, or both, compared to the LNP in the first or prior sample, optionally wherein the size (e.g., Rₕ) of the LNP in the second or subsequent sample is substantially identical to the size (e.g., Rₕ) of the LNP in the first or prior sample.

In some embodiments, the subject is a healthy subject. In some embodiments, the subject has, or is likely to have, a disorder, e.g., an infection, a cancer, or an autoimmune disorder. In some embodiments, the subject is a human. In some embodiments, the subject is an animal, e.g., a mammal, e.g., a mouse, a rat, or a primate.

In some embodiments, the SEC is performed in a single-column configuration. In some embodiments, the SEC is performed in a dual-column configuration, e.g., using two columns with different pore sizes, e.g., a first column with a first average pore size and a second column with a second average pore size. In some embodiments, the first average pore size is greater than the second average pore size. In some embodiments, the first average pore size has a molecular weight cut-off (MWCO) of about 1 MDa or more, e.g., about 2 MDa or more, 5 MDa or more, 10 MDa or more, 15 MDa or more, or 20 MDa or more. In some embodiments, the first average pore size has an MWCO of about 20 MDa or less, e.g., about 15 MDa or less, 10 MDa or less, 5 MDa or less, 2 MDa or less, or 1 MDa or less. In some embodiments, the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa. In some embodiments, the second average pore size has an MWCO of about 10 MDa or less, e.g., about 5 MDa or less, 2 MDa or less, 1 MDa or less, 0.5 MDa or less, 0.2 MDa or less, or 0.1 MDa or less. In some embodiments, the second average pore size has an MWCO of about 0.1 MDa or more, e.g., about 0.2 MDa or more, 0.5 MDa or more, 1 MDa or more, 2 MDa or more, 5 MDa or more, or 10 MDa or more. In some embodiments, the second average pore size has an MWCO of about 0.1 MDa to 10 MDa, e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.

In some embodiments, the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 250 mm.

In some embodiments, the SEC is performed using a polymer-based column. In some embodiments, the SEC is not performed using a silica-based column.

In some embodiments, the SEC is performed under an aqueous condition, e.g., with a bio-compatible buffer system, e.g., a phosphate buffer, a bicarbonate buffer, an acetate buffer, a Tris buffer, or any of the Good's buffers (e.g., as described in Good et al. Biochemistry. 1966; 5(2):467-77; Good and Izawa Methods Enzymol. 1972; 24:53-68; Ferguson et al. Anal Biochem. 1980 May; 104(2):300-10), e.g., any of MES, Bis-tris methane, ADA, Bis-tris propane, PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS, or a combination thereof. In some embodiments, the SEC is not performed sing an organic solvent (e.g., THF) as a mobile phase.

In some embodiments, the MALS is performed at a scattering angle of 15° and 90°. In some embodiments, the MALS is performed using a laser wavelength of about 500 nm to 800 nm, e.g., about 600 nm to 700 nm, 500 nm to 700 nm, 600 nm to 800 nm, 500 nm to 600 nm, 700 nm to 800 nm, e.g., about 658 nm. In some embodiments, the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL. In some embodiments, the MALS is performed using a scattering volume of about 0.001 μL to 0.1 μL, e.g., about 0.005 μL to 0.05 μL, 0.001 μL to 0.05 μL, 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL. In some embodiments, the MALS is performed at a temperature range of about 20° C. to 70° C., e.g., about 25° C. to 75° C. or 30° C. to 60° C. In some embodiments, the MALS is performed with a temperature stability of no more than ±1° C. e.g., no more than ±0.5° C. or 0.2° C. In some embodiments, the MALS is performed at a pH range of about 1-12, e.g., about 2-11 or 2-10.

In some embodiments, the size of the LNP is determined, e.g., the hydrodynamic radius (Rₕ) of the LNP is determined. In some embodiments, the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined. In some embodiments, the polydispersity index is determined, e.g., by calculating the ratio of M_{w} to Mₙ. In some embodiments, the R_{gw} of the LNP is determined, e.g., using a Zimm or partial Zimm approach (e.g., as described in Wyatt. Analytica Chimica Acta. 1993. 272: 1-40). In some embodiments, the half-life the LNP is determined. In some embodiments, the stability of the LNP is determined in accordance with a method described herein, e.g., a method described in Example 1.

In some embodiments, the method comprises comparing the acquired MALS signals directly. In some embodiments, the method comprises comparing the acquired MALS signals indirectly.

In some embodiments, a plurality of LNPs in the sample is analyzed, e.g., in accordance with a method described herein.

In some embodiments, the method does not comprise a step of recovering the LNP, e.g., by ultracentrifugation. In some embodiments, the method does not comprise a step of diluting the sample, e.g., to remove a high molecular weight component (e.g., plasma or serum component) that interferes with detection (e.g., detection by DLS). In some embodiments, the method does not comprises a step of labeling the LNP, e.g., with a fluorophore. In some embodiments, the method quantitatively detects the LNP in the sample.

In some embodiments, the LNP is loaded with a nucleic acid. In some embodiments, the LNP is not loaded with a nucleic acid. In some embodiments, the nucleic acid is a therapeutic nucleic acid (TNA). In some embodiments, the nucleic acid is an mRNA. In some embodiments, the nucleic acid is a vaccine. In some embodiments, the nucleic acid is a non-coding RNA, e.g., a small non-coding RNA. In some embodiments, the nucleic acid is a small interfering RNA (siRNA), an antisense oligonucleotide (ASO), or a microRNA (miRNA). In some embodiments, the nucleic acid is a guide RNA (gRNA). In some embodiments, the nucleic acid is a DNA. In some embodiments, the nucleic acid is an aptamer. In some embodiments, the nucleic acid comprises one or more modified nucleotides. In some embodiments, the nucleic acid is single stranded. In some embodiments, the nucleic acid is double stranded.

In some embodiments, the sample comprises a physiological fluid. In some embodiments, the physiological fluid is plasma. In some embodiments, the physiological fluid is serum. In some embodiments, the physiological fluid is blood. In some embodiments, the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9. In some embodiments, the physiological fluid has a pH of serum or plasma (e.g., 7.3-7.5). In some embodiments, the physiological fluid has a pH of a tumor microenvironment (e.g., pH 5.6 to 6.8).

In yet another aspect, the disclosure provides a method of determining the stability of a lipid nanoparticle (LNP) in a sample. The method comprises: (optionally) acquiring a first aliquot of the sample; subjecting the first aliquot to a size-exclusion chromatography (SEC): acquiring a multi angle light scattering (MALS) signal from the first aliquot: (optionally) acquiring a second aliquot of the sample; subjecting the second sample to the SEC; acquiring a second MALS signal from the second aliquot; and comparing the first MALS signal and the second MALS signal, wherein the comparison between the first MALS signal and the second MALS signal is indicative of the stability of the LNP in the sample, thereby determining the stability of the LNP in the sample.

In some embodiments, the second aliquot is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first aliquot is acquired.

In some embodiments, the method further comprises: (optionally) acquiring a third aliquot of the sample; subjecting the third sample to the SEC; acquiring a third MALS signal from the third aliquot; and comparing the third MALS signal with the first MALS signal, the second MALS signal, or both, wherein the comparison is indicative of the stability of the LNP in the sample. In some embodiments, the third aliquot is acquired after the second aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second aliquot is acquired.

In some embodiments, the method further comprises: (optionally) acquiring a fourth aliquot of the sample; subjecting the fourth sample to the SEC; acquiring a fourth MALS signal from the fourth aliquot; and comparing the fourth MALS signal with one or more (e.g., all) of the first MALS signal, the second MALS signal, or the third MALS signal, wherein the comparison is indicative of the stability of the LNP in the sample. In some embodiments, the fourth aliquot is acquired after the third aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third aliquot is acquired.

In some embodiments, the method further comprises: (optionally) acquiring a fifth aliquot of the sample; subjecting the fifth aliquot to the SEC; acquiring a fifth MALS signal from the fifth aliquot; and comparing the fourth MALS signal with one or more (e.g., all) of the first MALS signal, the second MALS signal, the third MALS signal, or the fourth MALS signal, wherein the comparison is indicative of the stability of the LNP in the sample. In some embodiments, the fifth aliquot is acquired after the fourth aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth aliquot is acquired.

In some embodiments, the LNP in the second or subsequent aliquot has a change to the LNP composition, e.g., lipid, nucleic acid, or both, compared to the LNP in the first or prior aliquot, optionally wherein the size (e.g., Rₕ) of the LNP in the second or subsequent aliquot is substantially identical to the size (e.g., Rn) of the LNP in the first or prior aliquot.

In some embodiments, the sample is not acquired from a subject, e.g., a physiological fluid reconstituted with the LNP. In some embodiments, the sample is acquired from a subject. In some embodiments, the subject is a healthy subject. In some embodiments, the subject has, or is likely to have, a disorder, e.g., an infection, a cancer, or an autoimmune disorder. In some embodiments, the subject is a human. In some embodiments, the subject is an animal, e.g., a mammal, e.g., a mouse, a rat, or a primate.

In still another aspect, the disclosure provides a method of determining the purity of a lipid nanoparticle (LNP) in a sample. The method comprises: (optionally) acquiring a first aliquot of the sample; subjecting the first aliquot to a size-exclusion chromatography (SEC); acquiring a multi angle light scattering (MALS) signal from the first aliquot; (optionally) acquiring a second aliquot of the sample; subjecting the second aliquot to the SEC; acquiring a second MALS signal from the second aliquot; and comparing the first MALS signal and the second MALS signal to determine the stability of the LNP in the sample, which is indicative of the purity of the LNP in the sample, thereby determining the purity of the LNP in the sample.

In some embodiments, the second aliquot is acquired after the first aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first aliquot is acquired.

In some embodiments, the method further comprises: (optionally) acquiring a first reference aliquot of a reference sample comprising an LNP; subjecting the first aliquot to the SEC; acquiring an MALS signal from the first reference aliquot; (optionally) acquiring a second reference aliquot of the reference sample; subjecting the second reference aliquot to the SEC: acquiring a second MALS signal from the second reference aliquot; and comparing the first MALS signal and the second MALS signal to determine the stability of the LNP in the reference sample.

In some embodiments, the method further comprises comparing the stability of the LNP in the sample with the stability of the LNP in the reference sample, thereby determining the purity of the LNP in the sample.

In some embodiments, the method further comprises: (optionally) acquiring a third aliquot of the sample (or the reference sample); subjecting the third aliquot to the SEC; acquiring a third MALS signal from the third aliquot; and comparing the third MALS signal with the first MALS signal, the second MALS signal, or both. In some embodiments, the third aliquot is acquired after the second aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second aliquot is acquired.

In some embodiments, the method further comprises: (optionally) acquiring a fourth aliquot of the sample (or the reference sample); subjecting the fourth aliquot to the SEC; acquiring a fourth MALS signal from the fourth aliquot; and comparing the fourth MALS signal with one or more (e.g., all) of the first MALS signal, the second MALS signal, or the third MALS signal. In some embodiments, the fourth aliquot is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third aliquot is acquired.

In some embodiments, the method further comprises: (optionally) acquiring a fifth aliquot of the sample (or the reference sample); subjecting the fifth sample to the SEC; acquiring a fifth MALS signal from the fifth aliquot; and comparing the fourth MALS signal with one or more (e.g., all) of the first MALS signal, the second MALS signal, the third MALS signal, or the fourth MALS signal. In some embodiments, the fifth aliquot is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth aliquot is acquired.

In some embodiments, the LNP in the sample has an impurity due to incompletely de-protected form of the LNP.

In some embodiments, the sample is not acquired from a subject, e.g., a physiological fluid reconstituted with the LNP. In some embodiments, the sample is acquired from a subject. In some embodiments, the subject is a healthy subject. In some embodiments, the subject has, or is likely to have, a disorder, e.g., an infection, a cancer, or an autoimmune disorder. In some embodiments, the subject is a human. In some embodiments, the subject is an animal, e.g., a mammal, e.g., a mouse, a rat, or a primate.

In some embodiments, the SEC is performed in a single-column configuration. In some embodiments, the SEC is performed in a dual-column configuration, e.g., using two columns with different pore sizes, e.g., a first column with a first average pore size and a second column with a second average pore size. In some embodiments, the first average pore size is greater than the second average pore size. In some embodiments, the first average pore size has a molecular weight cut-off (MWCO) of about 1 MDa or more, e.g., about 2 MDa or more, 5 MDa or more, 10 MDa or more, 15 MDa or more, or 20 MDa or more. In some embodiments, the first average pore size has an MWCO of about 20 MDa or less, e.g., about 15 MDa or less, 10 MDa or less, 5 MDa or less, 2 MDa or less, or 1 MDa or less. In some embodiments, the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa. In some embodiments, the second average pore size has an MWCO of about 10 MDa or less, e.g., about 5 MDa or less, 2 MDa or less, 1 MDa or less, 0.5 MDa or less, 0.2 MDa or less, or 0.1 MDa or less. In some embodiments, the second average pore size has an MWCO of about 0.1 MDa or more, e.g., about 0.2 MDa or more, 0.5 MDa or more, 1 MDa or more, 2 MDa or more, 5 MDa or more, or 10 MDa or more. In some embodiments, the second average pore size has an MWCO of about 0.1 MDa to 10 MDa, e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.

In some embodiments, the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 250 mm.

In some embodiments, the SEC is performed using a polymer-based column. In some embodiments, the SEC is not performed using a silica-based column.

In some embodiments, the SEC is performed under an aqueous condition, e.g., with a bio-compatible buffer system, e.g., a phosphate buffer, a bicarbonate buffer, an acetate buffer, a Tris buffer, or any of the Good's buffers (e.g., as described in Good et al. Biochemistry. 1966; 5(2):467-77; Good and Izawa Methods Enzymol. 1972:24:53-68; Ferguson et al. Anal Biochem. 1980 May; 104(2):300-10), e.g., any of MES, Bis-tris methane, ADA, Bis-tris propane, PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS, or a combination thereof. In some embodiments, the SEC is not performed sing an organic solvent (e.g., THF) as a mobile phase.

In some embodiments, the MALS is performed at a scattering angle of 15° and 90°. In some embodiments, the MALS is performed using a laser wavelength of about 500 nm to 800 nm, e.g., about 600 nm to 700 nm, 500 nm to 700 nm, 600 nm to 800 nm, 500 nm to 600 nm, 700 nm to 800 nm, e.g., about 658 nm. In some embodiments, the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL. In some embodiments, the MALS is performed using a scattering volume of about 0.001 μL to 0.1 μL, e.g., about 0.005 μL to 0.05 μL, 0.001 μL to 0.05 μL, 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL. In some embodiments, the MALS is performed at a temperature range of about 20° C. to 70° C., e.g., about 25° C. to 75° C. or 30° C. to 60° C. In some embodiments, the MALS is performed with a temperature stability of no more than ±1° C. e.g., no more than ±0.5° C. or 0.2° C. In some embodiments, the MALS is performed at a pH range of about 1-12, e.g., about 2-11 or 2-10.

In some embodiments, the size of the LNP is determined, e.g., the hydrodynamic radius (Rₕ) of the LNP is determined. In some embodiments, the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined. In some embodiments, the polydispersity index is determined, e.g., by calculating the ratio of M_{w} to Mₙ. In some embodiments, the R_{gw} of the LNP is determined, e.g., using a Zimm or partial Zimm approach (e.g., as described in Wyatt. Analytica Chimica Acta. 1993. 272: 1-40). In some embodiments, the half-life the LNP is determined. In some embodiments, the stability of the LNP is determined in accordance with a method described herein, e.g., a method described in Example 1.

In some embodiments, the method comprises comparing the acquired MALS signals directly. In some embodiments, the method comprises comparing the acquired MALS signals indirectly.

In some embodiments, a plurality of LNPs in the sample is analyzed, e.g., in accordance with a method described herein.

In some embodiments, the method does not comprise a step of recovering the LNP, e.g., by ultracentrifugation. In some embodiments, the method does not comprise a step of diluting the sample, e.g., to remove a high molecular weight component (e.g., plasma or serum component) that interferes with detection (e.g., detection by DLS). In some embodiments, the method does not comprises a step of labeling the LNP, e.g., with a fluorophore. In some embodiments, the method quantitatively detects the LNP in the sample.

In some embodiments, the LNP is loaded with a nucleic acid. In some embodiments, the LNP is not loaded with a nucleic acid. In some embodiments, the nucleic acid is a therapeutic nucleic acid (TNA). In some embodiments, the nucleic acid is an mRNA. In some embodiments, the nucleic acid is a vaccine. In some embodiments, the nucleic acid is a non-coding RNA, e.g., a small non-coding RNA. In some embodiments, the nucleic acid is a small interfering RNA (siRNA), an antisense oligonucleotide (ASO), or a microRNA (miRNA). In some embodiments, the nucleic acid is a guide RNA (gRNA). In some embodiments, the nucleic acid is a DNA. In some embodiments, the nucleic acid is an aptamer. In some embodiments, the nucleic acid comprises one or more modified nucleotides. In some embodiments, the nucleic acid is single stranded. In some embodiments, the nucleic acid is double stranded.

In some embodiments, the sample comprises a physiological fluid. In some embodiments, the physiological fluid is plasma. In some embodiments, the physiological fluid is serum. In some embodiments, the physiological fluid is blood. In some embodiments, the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9. In some embodiments, the physiological fluid has a pH of serum or plasma (e.g., 7.3-7.5). In some embodiments, the physiological fluid has a pH of a tumor microenvironment (e.g., pH 5.6 to 6.8).

In another aspect, the disclosure provides a method of monitoring a process of manufacturing a lipid nanoparticle (LNP). The method comprises: (optionally) acquiring a first sample comprising an LNP from the process of manufacturing the LNP; subjecting the first sample to a size-exclusion chromatography (SEC); and acquiring a multi angle light scattering (MALS) signal from the first sample, thereby monitoring the process of manufacturing the LNP.

In some embodiments, the method further comprises: (optionally) acquiring a second sample comprising the LNP from the process of manufacturing the LNP; subjecting the second sample to the SEC; and acquiring a second MALS signal from the second sample. In some embodiments, the second sample is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first sample is acquired.

In some embodiments, the method further comprises: (optionally) acquiring a third sample comprising the LNP from the process of manufacturing the LNP; subjecting the third sample to the SEC; and acquiring a third MALS signal from the third sample. In some embodiments, the third sample is acquired after the second sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second sample is acquired.

In some embodiments, the method further comprises: (optionally) acquiring a fourth sample comprising the LNP from the process of manufacturing the LNP: subjecting the fourth sample to the SEC; and acquiring a fourth MALS signal from the fourth sample. In some embodiments, the fourth sample is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third sample is acquired.

In some embodiments, the method further comprises: (optionally) acquiring a fifth sample comprising the LNP from the process of manufacturing the LNP; subjecting the fifth sample to the SEC; and acquiring a fifth MALS signal from the fifth sample. In some embodiments, the fifth sample is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth sample is acquired.

In some embodiments, the SEC is performed in a single-column configuration. In some embodiments, the SEC is performed in a dual-column configuration, e.g., using two columns with different pore sizes, e.g., a first column with a first average pore size and a second column with a second average pore size. In some embodiments, the first average pore size is greater than the second average pore size. In some embodiments, the first average pore size has a molecular weight cut-off (MWCO) of about 1 MDa or more, e.g., about 2 MDa or more, 5 MDa or more, 10 MDa or more, 15 MDa or more, or 20 MDa or more. In some embodiments, the first average pore size has an MWCO of about 20 MDa or less, e.g., about 15 MDa or less, 10 MDa or less, 5 MDa or less, 2 MDa or less, or 1 MDa or less. In some embodiments, the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa. In some embodiments, the second average pore size has an MWCO of about 10 MDa or less, e.g., about 5 MDa or less, 2 MDa or less, 1 MDa or less, 0.5 MDa or less, 0.2 MDa or less, or 0.1 MDa or less. In some embodiments, the second average pore size has an MWCO of about 0.1 MDa or more, e.g., about 0.2 MDa or more, 0.5 MDa or more, 1 MDa or more, 2 MDa or more, 5 MDa or more, or 10 MDa or more. In some embodiments, the second average pore size has an MWCO of about 0.1 MDa to 10 MDa, e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.

In some embodiments, the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 250 mm.

In some embodiments, the SEC is performed using a polymer-based column. In some embodiments, the SEC is not performed using a silica-based column.

In some embodiments, the SEC is performed under an aqueous condition, e.g., with a bio-compatible buffer system, e.g., a phosphate buffer, a bicarbonate buffer, an acetate buffer, a Tris buffer, or any of the Good's buffers (e.g., as described in Good et al. Biochemistry. 1966; 5(2):467-77; Good and Izawa Methods Enzymol. 1972; 24:53-68; Ferguson et al. Anal Biochem. 1980 May; 104(2):300-10), e.g., any of MES, Bis-tris methane, ADA, Bis-tris propane, PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS, or a combination thereof. In some embodiments, the SEC is not performed sing an organic solvent (e.g., THF) as a mobile phase.

In some embodiments, the MALS is performed at a scattering angle of 15° and 90°. In some embodiments, the MALS is performed using a laser wavelength of about 500 nm to 800 nm, e.g., about 600 nm to 700 nm, 500 nm to 700 nm, 600 nm to 800 nm, 500 nm to 600 nm, 700 nm to 800 nm, e.g., about 658 nm. In some embodiments, the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL. In some embodiments, the MALS is performed using a scattering volume of about 0.001 μL to 0.1 μL, e.g., about 0.005 μL to 0.05 μL, 0.001 μL to 0.05 μL, 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL. In some embodiments, the MALS is performed at a temperature range of about 20° C. to 70° C., e.g., about 25° C. to 75° C. or 30° C. to 60° C. In some embodiments, the MALS is performed with a temperature stability of no more than f 1° C., e.g., no more than: 0.5° C. or 0.2° C. In some embodiments, the MALS is performed at a pH range of about 1-12, e.g., about 2-11 or 2-10.

In some embodiments, the size of the LNP is determined, e.g., the hydrodynamic radius (Rₕ) of the LNP is determined. In some embodiments, the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined. In some embodiments, the polydispersity index is determined, e.g., by calculating the ratio of M_{w} to Mₙ. In some embodiments, the R_{gw} of the LNP is determined, e.g., using a Zimm or partial Zimm approach (e.g., as described in Wyatt. Analytica Chimica Acta. 1993. 272: 1-40). In some embodiments, the half-life the LNP is determined. In some embodiments, the stability of the LNP is determined in accordance with a method described herein, e.g., a method described in Example 1.

In some embodiments, the method comprises comparing the acquired MALS signals directly. In some embodiments, the method comprises comparing the acquired MALS signals indirectly.

In some embodiments, a plurality of LNPs in the sample is analyzed, e.g., in accordance with a method described herein.

In some embodiments, the method does not comprise a step of recovering the LNP, e.g., by ultracentrifugation. In some embodiments, the method does not comprise a step of diluting the sample, e.g., to remove a high molecular weight component (e.g., plasma or serum component) that interferes with detection (e.g., detection by DLS). In some embodiments, the method does not comprises a step of labeling the LNP, e.g., with a fluorophore. In some embodiments, the method quantitatively detects the LNP in the sample.

In some embodiments, the LNP is loaded with a nucleic acid. In some embodiments, the LNP is not loaded with a nucleic acid. In some embodiments, the nucleic acid is a therapeutic nucleic acid (TNA). In some embodiments, the nucleic acid is an mRNA. In some embodiments, the nucleic acid is a vaccine. In some embodiments, the nucleic acid is a non-coding RNA, e.g., a small non-coding RNA. In some embodiments, the nucleic acid is a small interfering RNA (siRNA), an antisense oligonucleotide (ASO), or a microRNA (miRNA). In some embodiments, the nucleic acid is a guide RNA (gRNA). In some embodiments, the nucleic acid is a DNA. In some embodiments, the nucleic acid is an aptamer. In some embodiments, the nucleic acid comprises one or more modified nucleotides. In some embodiments, the nucleic acid is single stranded. In some embodiments, the nucleic acid is double stranded.

In some embodiments, the sample comprises a physiological fluid. In some embodiments, the physiological fluid is plasma. In some embodiments, the physiological fluid is serum. In some embodiments, the physiological fluid is blood. In some embodiments, the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9. In some embodiments, the physiological fluid has a pH of serum or plasma (e.g., 7.3-7.5). In some embodiments, the physiological fluid has a pH of a tumor microenvironment (e.g., pH 5.6 to 6.8).

In yet another aspect, the disclosure provides a method of determining the pharmacokinetics (PK) of a lipid nanoparticle (LNP). The method comprises: (optionally) acquiring a first sample comprising an LNP from a subject; subjecting the first sample to a size-exclusion chromatography (SEC); and acquiring a multi angle light scattering (MALS) signal from the first sample, thereby determining the PK of the LNP.

In some embodiments, the method further comprises: (optionally) acquiring a second sample comprising the LNP from the subject: subjecting the second sample to the SEC: and acquiring a second MALS signal from the second sample. In some embodiments, the second sample is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first sample is acquired.

In some embodiments, the method further comprises: (optionally) acquiring a third sample comprising the LNP from the subject: subjecting the third sample to the SEC; and acquiring a third MALS signal from the third sample. In some embodiments, the third sample is acquired after the second sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second sample is acquired.

In some embodiments, the method further comprises: (optionally) acquiring a fourth sample comprising the LNP from the subject: subjecting the fourth sample to the SEC; and acquiring a fourth MALS signal from the fourth sample. In some embodiments, the fourth sample is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third sample is acquired.

In some embodiments, the method further comprises: (optionally) acquiring a fifth sample comprising the LNP from the subject; subjecting the fifth sample to the SEC; and acquiring a fifth MALS signal from the fifth sample. In some embodiments, the fifth sample is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth sample is acquired.

In some embodiments, the subject is a healthy subject. In some embodiments, the subject has, or is likely to have, a disorder, e.g., an infection, a cancer, or an autoimmune disorder. In some embodiments, the subject is a human. In some embodiments, the subject is an animal, e.g., a mammal, e.g., a mouse, a rat, or a primate.

In some embodiments, the SEC is performed in a single-column configuration. In some embodiments, the SEC is performed in a dual-column configuration, e.g., using two columns with different pore sizes, e.g., a first column with a first average pore size and a second column with a second average pore size. In some embodiments, the first average pore size is greater than the second average pore size. In some embodiments, the first average pore size has a molecular weight cut-off (MWCO) of about 1 MDa or more, e.g., about 2 MDa or more, 5 MDa or more, 10 MDa or more, 15 MDa or more, or 20 MDa or more. In some embodiments, the first average pore size has an MWCO of about 20 MDa or less, e.g., about 15 MDa or less, 10 MDa or less, 5 MDa or less, 2 MDa or less, or 1 MDa or less. In some embodiments, the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa. In some embodiments, the second average pore size has an MWCO of about 10 MDa or less, e.g., about 5 MDa or less, 2 MDa or less, 1 MDa or less, 0.5 MDa or less, 0.2 MDa or less, or 0.1 MDa or less. In some embodiments, the second average pore size has an MWCO of about 0.1 MDa or more, e.g., about 0.2 MDa or more, 0.5 MDa or more, 1 MDa or more, 2 MDa or more, 5 MDa or more, or 10 MDa or more. In some embodiments, the second average pore size has an MWCO of about 0.1 MDa to 10 MDa, e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.

In some embodiments, the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 250 mm.

In some embodiments, the SEC is performed using a polymer-based column. In some embodiments, the SEC is not performed using a silica-based column.

In some embodiments, the SEC is performed under an aqueous condition, e.g., with a bio-compatible buffer system, e.g., a phosphate buffer, a bicarbonate buffer, an acetate buffer, a Tris buffer, or any of the Good's buffers (e.g., as described in Good et al. Biochemistry. 1966; 5(2):467-77; Good and Izawa Methods Enzymol. 1972; 24:53-68; Ferguson et al. Anal Biochem. 1980 May; 104(2):300-10), e.g., any of MES, Bis-tris methane, ADA, Bis-tris propane, PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS, or a combination thereof. In some embodiments, the SEC is not performed sing an organic solvent (e.g., THF) as a mobile phase.

In some embodiments, the MALS is performed at a scattering angle of 15° and 90°. In some embodiments, the MALS is performed using a laser wavelength of about 500 nm to 800 mun, e.g., about 600 nm to 700 nm, 500 nm to 700 nm, 600 nm to 800 nm, 500 nm to 600 nm, 700 nm to 800 nm, e.g., about 658 nm. In some embodiments, the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL. In some embodiments, the MALS is performed using a scattering volume of about 0.001 μL to 0.1 μL, e.g., about 0.005 μL to 0.05 μL, 0.001 μL to 0.05 μL, 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL. In some embodiments, the MALS is performed at a temperature range of about 20° C. to 70° C., e.g., about 25° C. to 75° C. or 30° C. to 60° C. In some embodiments, the MALS is performed with a temperature stability of no more than ±1° C., e.g., no more than ±0.5° C. or 0.2° C. In some embodiments, the MALS is performed at a pH range of about 1-12, e.g., about 2-11 or 2-10.

In some embodiments, the size of the LNP is determined, e.g., the hydrodynamic radius (Rₕ) of the LNP is determined. In some embodiments, the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined. In some embodiments, the polydispersity index is determined, e.g., by calculating the ratio of M_{w} to Mₙ. In some embodiments, the R_{gw} of the LNP is determined, e.g., using a Zimm or partial Zimm approach (e.g., as described in Wyatt. Analytica Chimica Acta. 1993. 272: 1-40). In some embodiments, the half-life the LNP is determined. In some embodiments, the stability of the LNP is determined in accordance with a method described herein, e.g., a method described in Example 1.

In some embodiments, the method comprises comparing the acquired MALS signals directly. In some embodiments, the method comprises comparing the obtained MALS signals indirectly.

In some embodiments, a plurality of LNPs in the sample is analyzed, e.g., in accordance with a method described herein.

In some embodiments, the method does not comprise a step of recovering the LNP, e.g., by ultracentrifugation. In some embodiments, the method does not comprise a step of diluting the sample, e.g., to remove a high molecular weight component (e.g., plasma or serum component) that interferes with detection (e.g., detection by DLS). In some embodiments, the method does not comprises a step of labeling the LNP, e.g., with a fluorophore. In some embodiments, the method quantitatively detects the LNP in the sample.

In some embodiments, the LNP is loaded with a nucleic acid. In some embodiments, the LNP is not loaded with a nucleic acid. In some embodiments, the nucleic acid is a therapeutic nucleic acid (TNA). In some embodiments, the nucleic acid is an mRNA. In some embodiments, the nucleic acid is a vaccine. In some embodiments, the nucleic acid is a non-coding RNA, e.g., a small non-coding RNA. In some embodiments, the nucleic acid is a small interfering RNA (siRNA), an antisense oligonucleotide (ASO), or a microRNA (miRNA). In some embodiments, the nucleic acid is a guide RNA (gRNA). In some embodiments, the nucleic acid is a DNA. In some embodiments, the nucleic acid is an aptamer. In some embodiments, the nucleic acid comprises one or more modified nucleotides. In some embodiments, the nucleic acid is single stranded. In some embodiments, the nucleic acid is double stranded.

In some embodiments, the sample comprises a physiological fluid. In some embodiments, the physiological fluid is plasma. In some embodiments, the physiological fluid is serum. In some embodiments, the physiological fluid is blood. In some embodiments, the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit. In some embodiments, the physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9. In some embodiments, the physiological fluid has a pH of serum or plasma (e.g., 7.3-7.5). In some embodiments, the physiological fluid has a pH of a tumor microenvironment (e.g., pH 5.6 to 6.8).

In another aspect, the disclosure provides a system comprising a processor configured to perform a method described herein.

In some embodiments, the processor is configured to subject a sample or an aliquot of a sample to a size-exclusion chromatography (SEC). In some embodiments, the processor is further configured to obtain a multi angle light scattering (MALS) signal from the sample or the aliquot of the sample.

### BRIEF DESCRIPTION OF THE DRAWINGS

FIG. 1A is a diagram depicting exemplary mRNA-LNP detection using an SEC-MALS pipeline.

FIG. 1B is an SEC-MALS chromatogram of mRNA-LNPs in 50 mM phosphate buffer (pH 7.2). The mRNA-LNP peak is marked “1” at its base. Dissolved air and buffer salts caused artifacts in the RI trace that were well-separated from the mRNA-LNP peak and did not impact the calculated M_{w} and R_{gw} values.

FIGS. 2A-2D depict the separation of human plasma from mRNA-LNPs by dual-column SEC. LS90° (FIGS. 2A-2B) and Refractive Index (FIGS. 2C-2D) chromatograms show minimal interference of plasma components with the mRNA-LNP peak in the dual-column configuration.

FIGS. 3A-3D depicts the degradation of mRNA-LNPs in human plasma. LS90° (FIG. 3A), UV absorbance at 260 nm (FIG. 3B), and Refractive index (FIG. 3C) chromatograms of mRNA-LNPs in human plasma at room temperature are shown. 30 μl injections were performed at 15 min intervals. FIGS. 3B-3C insets show zoomed representations of the mRNA-LNP peak. Overlaid online dynamic light scattering signals from all injections are shown in FIG. 3D.

FIG. 4A-4D depicts LNP degradation kinetics and physical characteristics revealed by MALS. Kinetics of mRNA-LNP and empty LNP degradation in human plasma and serum tracked by LS90° peak area are shown in FIG. 4A. FIG. 4B shows that mRNA-LNPs maintain a constant apparent Mw in serum, but not in plasma. FIGS. 4C-4D show that mRNA-LNPs maintain a constant Rgw in serum, but not in plasma.

FIGS. 5A-5C depict LC-MS/MS quantitation of mRNA-LNP composition during serum and plasma degradation. Overlaid PRM chromatograms of Cholesterol (369.65→147.16), DSPC (790.82→184.13), ALC-0315 (766.91→510.62), and ALC-0159 (1184.10→494.62) are shown in FIG. 5A. Quantities injected: 1, 2, 4 and 8 pmol of DSPC, ALC-0315 and ALC-0159; 10, 20, 40 and 80 pmol of Cholesterol. See FIGS. 9A-9D for the mass spectra of each compound. Compositions of mRNA-LNPs degrading in serum and plasma are shown in FIG. 5B-5C.

FIGS. 6A-6D depict the reduction of mRNA-LNP stability in serum by a lipid impurity in the ionizable lipid. Total ion chromatograms of lipid components from mRNA-LNPs without impurity (FIG. 6A) and with impurity (FIG. 6B) are shown. The impurity peak elutes at 6.5 mins and is marked with a red triangle. Cholesterol is shown as extracted ion chromatograms shown inset. Mass spectrum of impurity peak with isotopic resolution shown inset is shown in FIG. 6C. Degradation of mRNA-LNPs with and without impurity in serum is shown in FIG. 6D.

FIGS. 7A-7B depict the molecular structure of ALC-0315 (FIG. 7A) and the putative molecular structure of impurity (FIG. 7B), respectively. Theoretical monoisotopic masses are shown.

FIG. 8 depicts ALC-0315 impurity containing O-Boc protection group. Extracted ion chromatograms of m/z=766.72 and 866.82 for uncontaminated ALC-0315 (top), ALC-0315 impurity fraction (middle), ALC-0315 impurity fraction subjected to deprotection (bottom), are shown. The low sample pH caused slight retention time shifts in panel (bottom).

FIGS. 9A-9D depict the MS/MS spectra of Cholesterol (FIG. 9A), DSPC (FIG. 9B), ALC-0315 (FIG. 9C) and ALC-0159 (FIG. 9D). MS1 spectra are shown inset (FIGS. 9B, 9D) where the precursor ions are not apparent in the MS/MS spectrum.

FIGS. 10-10D are representative calibration curves for Cholesterol (FIG. 10A), DSPC (FIG. 10B), ALC-0315 (FIG. 10C) and ALC-0159 (FIG. 10D).

FIG. 11A is an LC-UV chromatogram of contaminated batch of ALC-0315. Absorbance at 215 nm was used to monitor ester groups in the eluting compounds, and for relative quantitation of ALC-0315 (RT=15.48 min) and contaminants (RT=17.66-24.86 min). FIG. 11B depicts the MS1 of ALC-0315 peak showing only a trace amount of impurity (866.9 m/z, red arrow). FIG. 11C depicts the MS1 of contaminants showing a far stronger impurity signal (866.9 m/z, red arrow).

FIG. 12A is an LC-UV chromatogram of contaminated batch of ALC-0315. Absorbance at 215 nm was used to monitor ester groups in the eluting compounds, and for relative quantitation of ALC-0315 (RT=14.96 min) and contaminants (RT=17.60-25.16 min). FIG. 12B depicts the MS1 of ALC-0315 peak. FIG. 12C depicts the MS1 of contaminants. No significant impurity signal at 866.9 m/z was seen in MS1 of the main peak (FIG. 12B) and contaminants (FIG. 12C).

### DETAILED DESCRIPTION

The disclosure is based, at least in part, the discovery that size-exclusion chromatography coupled with multi-angle light scattering (SEC-MALS) is a highly effective tool for quantitatively characterizing the stability of LNPs in physiological fluids. Optimized chromatography permits separation of LNPs from interfering plasma and serum components, allowing for both reaction kinetics and changes to nanoparticle physical properties to be accurately characterized.

Many emerging LNP drugs may require systemic administration of LNPs instead of the more localized intramuscular injection. As intravenous injection brings LNPs into contact with human blood, the stability of LNPs in human plasma or serum is an important factor in drug development as this is expected to influence both efficacy and pharmacokinetics.

However, current methods to measure LNP stability after exposure to human plasma or serum have several drawbacks. Most methods rely on Dynamic Light Scattering (DLS) to measure the size profile of LNPs after recovery by ultracentrifugation or gel filtration to establish stability. In some cases, DLS is used to directly measure the size profile of LNPs in dilute serum solutions. Unfortunately, DLS cannot accurately determine the number of intact LNPs over a period of time, which is the most direct measure of stability. In general, DLS can only measure size profiles and not the number of particles of any given size.

Concretely, DLS-based methods may consider an LNP sample stable if almost all recovered LNPs are close to the original size, even if fewer LNPs are recovered due to an ongoing degradation process. In principle, the increased polydispersity of LNPs could indicate degradation, but in practice this may be confounded by the inability of ultracentrifugation or gel filtration to efficiently recover partially degraded LNPs due to their greater buoyancy or smaller hydrodynamic radius (Rₕ).

In addition, LNP compositions may be altered by plasma components, which may be expected to change their properties despite possibly retaining their original Rₕ. Unfortunately, DLS provides little insight into such processes.

The methods described herein use size-exclusion chromatography coupled with multi-angle light scattering (SEC-MALS) to quantitatively study the stability of a widely used mRNA-LNP formulation in the presence of human plasma or serum. By separating mRNA-LNPs from interfering plasma and serum components, both the reaction kinetics and changes to nanoparticle physical properties can be accurately characterized. Furthermore, the effect of trace contaminants in lipid excipients on the stability of mRNA-LNPs can be investigated.

This disclosure offers methods with improved accuracy for measuring LNP stability in physiological fluids. After separating LNPs from interfering plasma or serum components by SEC, MALS can be used to measure the number of intact LNPs remaining with high sensitivity based on their retention time. Degradation kinetics are dependent on the milieu (purified serum albumin vs. plasma/serum) and on the characteristics of the LNP (containing mRNA vs. empty). Based on measured biophysical parameters (R_{gw} and apparent M_{w}), SEC-MALS may reveal a progressive replacement of LNP lipids with plasma components (possibly proteins), which can be corroborated by LC-MS/MS. Thus, the methods described herein are useful for optimizing LNP formulations. The methods described herein can also detect the destabilization of mRNA-LNPs by impurities, demonstrating its utility as a quality control tool.

### Definitions

Unless defined otherwise, all technical and scientific terms used herein have the same meaning as commonly understood by one of ordinary skill in the art to which the invention pertains.

As used herein, the articles “a” and “an” refer to one or to more than one (e.g., to at least one) of the grammatical object of the article.

“About” and “approximately” as the term used herein shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the term used herein refers to acquiring possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent. In an embodiment, directly acquiring encompasses a direct measurement. In an embodiment, indirectly acquiring encompasses an inference.

“Acquiring a sample” as the term used herein refers to acquiring possession of a sample, e.g., a sample described herein, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third-party laboratory that directly acquired the sample). Directly acquiring a sample includes performing a process that includes a physical change in a physical substance, e.g., a starting material, such as a tissue, e.g., a tissue in a human patient or a tissue that was previously isolated from a patient. Exemplary changes include making a physical entity from a starting material; dissecting or scraping a tissue; separating or purifying a substance; combining two or more separate entities into a mixture; or performing a chemical reaction that includes breaking or forming a covalent or non-covalent bond.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

“Sample” as the term used herein refers to a biological sample obtained or derived from a source of interest. In an embodiment, the source of interest comprises an organism, such as an animal or human. The source of the sample can be blood or a blood constituent; a bodily fluid; a solid tissue as from a fresh, frozen and/or preserved organ, tissue, biopsy, resection, smear, or aspirate; or cells from any time in gestation or development of a subject. In an embodiment, the source of the sample is blood or a blood constituent. In an embodiment, the sample is a primary sample, e.g., obtained directly from a source of interest by any appropriate means. In an embodiment, the sample is a preparation that is obtained by processing (e.g., by removing one or more components of and/or by adding one or more agents to) a primary sample.

“Subject” as the term used herein is intended to include human and non-human animals. In some embodiments, the subject is a human subject, e.g., a healthy human subject or a human patient having a disorder, or at risk of having a disorder. The term “non-human animals” includes mammals and non-mammals, such as non-human primates.

### Size Exclusion Chromatography (SEC)

The methods and systems described herein can include size exclusion chromatography (SEC).

SEC, also sometimes known as molecular sieve chromatography, is a chromatographic method in which molecules in solution are generally separated by their size. Typically, when an aqueous solution is used to transport the sample through the column, the technique is known as gel-filtration chromatography, which uses spherical beads containing pores of a specific size distribution. Separation generally occurs when molecules of different sizes are included or excluded from the pores within the matrix. Smaller molecules diffuse into the pores and their flow through the column is retarded according to their size, while larger molecules do not enter the pores and are eluted in the column's void volume. Thus, molecules separate based on their size as they pass through the column and are eluted in order of decreasing molecular weight (MW).

The SEC can be performed in a single-column configuration or a multi-column configuration, e.g., using multiple columns with different pore sizes. In some embodiments, the SEC is performed in a dual-column configuration, e.g., using two columns with different pore sizes. For example, the SEC can be performed using a first column with a first average pore size and a second column with a second average pore size.

In some embodiments, the first average pore size is greater than the second average pore size. In some embodiments, the first average pore size has a molecular weight cut-off (MWCO) of about 1 MDa or more, e.g., about 2 MDa or more, 5 MDa or more, 10 MDa or more, 15 MDa or more, or 20 MDa or more. In some embodiments, the first average pore size has an MWCO of about 20 MDa or less, e.g., about 15 MDa or less, 10 MDa or less, 5 MDa or less, 2 MDa or less, or 1 MDa or less. In some embodiments, the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa.

In some embodiments, the second average pore size has an MWCO of about 10 MDa or less, e.g., about 5 MDa or less, 2 MDa or less, 1 MDa or less, 0.5 MDa or less, 0.2 MDa or less, or 0.1 MDa or less. In some embodiments, the second average pore size has an MWCO of about 0.1 MDa or more, e.g., about 0.2 MDa or more, 0.5 MDa or more, 1 MDa or more, 2 MDa or more, 5 MDa or more, or 10 MDa or more. In some embodiments, the second average pore size has an MWCO of about 0.1 MDa to 10 MDa, e.g., about 0.2 MDa to 5 MDa, 0.5 MDa to 2 MDa, 0.1 MDa to 5 MDa. 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.

In some embodiments, the first average pore size has an MWCO of about 1 MDa or more (e.g., about 2 MDa or more, 5 MDa or more, 10 MDa or more, 15 MDa or more, or 20 MDa or more) and the second average pore size has an MWCO of about 10 MDa or less (e.g., about 5 MDa or less, 2 MDa or less, 1 MDa or less, 0.5 MDa or less, 0.2 MDa or less, or 0.1 MDa or less), and the first average pore size is greater than the second average pore size. In some embodiments, the first average pore size has an MWCO of about 1 MDa to 20 MDa (e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa) and the second average pore size has an MWCO of about 0.1 MDa to 10 MDa (e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa), and the first average pore size is greater than the second average pore size.

In some embodiments, the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 200 mm, 250 mm, 300 mm, 400 mm, 500 mm, 600 mm, 700 mm, 800 mm, 900 mm, or 1000 mm.

In some embodiments, the SEC is performed using a polymer-based column. In some embodiments, the SEC is not performed using a polymer-based column. In some embodiments, the SEC is not performed using a silica-based column. In some embodiments, the SEC is performed using a silica-based column.

In some embodiments, the SEC is performed under an aqueous condition, e.g., with a bio-compatible buffer system. Exemplary buffers include, but are not limited to, a phosphate buffer, a bicarbonate buffer, an acetate buffer, or a Tris buffer. Other exemplary buffers include any of the Good's buffers, as described in Good et al. Biochemistry. 1966; 5(2):467-77; Good and Izawa Methods Enzymol. 1972:24:53-68; Ferguson et al. Anal Biochem. 1980; 104(2):300-10. For example, the buffer can be any of MES, Bis-tris methane, ADA, Bis-tris propane, PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris. Glycinamide. Glycylglycine, HEPBS, Bicine, TAPS, AMPB, CHES, CAPSO, AMP, CAPS, CABS, or a combination thereof.

In some embodiments, the SEC is not performed using an organic solvent (e.g., tetrahydrofuran or THF) as a mobile phase.

### Multi Angle Light Scattering (MALS)

The methods and systems described herein can use multi angle light scattering.

Multi angle light scattering (MALS), also sometimes known as multiangle light scattering or multi-angle light scattering, is a technique for measuring the light scattered by a sample into a plurality of angles. It can be used for determining both the absolute molar mass and the average size of molecules in solution, by detecting how they scatter light. A collimated beam from a laser source is most often used, in which case the technique can be referred to as multiangle laser light scattering (MALLS). The “multi-angle” term refers to the detection of scattered light at different discrete angles as measured, for example, by a single detector moved over a range that includes the particular angles selected or an array of detectors fixed at specific angular locations.

In some embodiments, the MALS is performed at two scattering angles that differ by 15°, 30°, 45°, 60°, 75°, or 90°. In some embodiments, the MALS is performed at a scattering angle of about 15° and about 90°. In some embodiments, the MALS is performed using a laser wavelength of about 500 nm to 800 nm, e.g., about 600 nm to 700 nm, 500 nm to 700 nm, 600 nm to 800 nm, 500 nm to 600 nm, 700 nm to 800 nm, e.g., about 658 nm. In some embodiments, the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL. In some embodiments, the MALS is performed using a scattering volume of about 0.001 μL to 0.1 μL, e.g., about 0.005 μL to 0.05 μL, 0.001 μL to 0.05 μL, 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL. In some embodiments, the MALS is performed at a temperature range of about 20° C. to 70° C., e.g., about 25° C. to 75° C. or 30° C. to 60 GC. In some embodiments, the MALS is performed with a temperature stability of no more than ±1° C., e.g., no more than ±0.5° C., ±0.4° C., ±0.3° C., ±0.2° C., or ±0.1° C. In some embodiments, the MALS is performed at a pH range of about 1-12, e.g., about 2-11, 2-10, 3-10, 4-9, 5-8, or 6-7.

In some embodiments, the size of the LNP is determined, e.g., the hydrodynamic radius (Rₕ) of the LNP is determined. In some embodiments, the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined. In some embodiments, the polydispersity index is determined, e.g., by calculating the ratio of M_{w} to Mₙ. In some embodiments, the R_{gw} of the LNP is determined, e.g., using a Zimm or partial Zimm approach (e.g., as described in Wyatt. Analytica Chimica Acta. 1993. 272: 1-40). In some embodiments, the half-life the LNP is determined. In some embodiments, the stability of the LNP is determined in accordance with a method described herein, e.g., a method described in Example 1.

In some embodiments, the MALS signals obtained in any of the methods described herein are compared directly. In some embodiments, the MALS signals obtained in any of the methods described herein are compared indirectly.

### Lipid Nanoparticle (LNP)

The methods and systems described herein can be used to analyze lipid nanoparticles.

Lipid nanoparticles (LNPs), are nanoparticles composed of lipids. An LNP is typically spherical with an average diameter between 10 and 1000 nanometers. Solid lipid nanoparticles possess a solid lipid core matrix that can solubilize lipophilic molecules. The lipid core is stabilized by surfactants (emulsifiers). The emulsifier used depends on administration routes and is more limited for parenteral administrations. The term lipid is used here in a broader sense and includes triglycerides (e.g. tristearin), diglycerides (e.g. glycerol behenate), monoglycerides (e.g. glycerol monostearate), fatty acids (e.g. stearic acid), steroids (e.g. cholesterol), and waxes (e.g. cetyl palmitate). All classes of emulsifiers (with respect to charge and molecular weight) can be used to stabilize the lipid dispersion. In some cases, the combination of emulsifiers may prevent particle agglomeration more efficiently. For example, LNPs used in certain mRNA vaccines can be made of four types of lipids: an ionizable cationic lipid (whose positive charge binds to negatively charged mRNA), a PEGylated lipid (for stability), a phospholipid (for structure), and cholesterol (for structure).

In some embodiments, the method does not comprise one or more (e.g., two or all) of the following steps: a step of recovering the LNP, e.g., by ultracentrifugation; a step of diluting the sample, e.g., to remove a high molecular weight component (e.g., plasma or serum component) that interferes with detection (e.g., detection by DLS); or a step of labeling the LNP, e.g., with a fluorophore. In some embodiments, LNP is not recovered, e.g., by ultracentrifugation. In some embodiments, the LNP is not diluted, e.g., to remove a high molecular weight component (e.g., plasma or serum component) that interferes with detection (e.g., detection by DLS). In some embodiments, the LNP is not labeled, e.g., with a fluorophore.

In some embodiments, the LNP is loaded with a nucleic acid. In some embodiments, the LNP is not loaded with a nucleic acid. In some embodiments, the nucleic acid is a therapeutic nucleic acid (TNA). In some embodiments, the nucleic acid is an mRNA. In some embodiments, the nucleic acid is a vaccine. In some embodiments, the nucleic acid is a non-coding RNA, e.g., a small non-coding RNA. In some embodiments, the nucleic acid is a small interfering RNA (siRNA), an antisense oligonucleotide (ASO), or a microRNA (miRNA). In some embodiments, the nucleic acid is a DNA. In some embodiments, the nucleic acid is an aptamer. In some embodiments, the nucleic acid comprises one or more modified nucleotides. In some embodiments, the nucleic acid is single stranded. In some embodiments, the nucleic acid is double stranded.

In some embodiments, a plurality of LNPs in the sample is analyzed, e.g., in accordance with a method described herein. In some embodiments, at least 50%, 60%, 70%, 80%, 90%, or 90% of the LNPs in the sample are loaded with a nucleic acid (e.g., a nucleic acid described herein). In some embodiments, the purity of the LNPs in the sample is at least 90%, 91%, 92%, 93%, 94%, 95%, 96%, 97%, 98%, 99%, or more. In some embodiments, the method quantitatively detects the LNPs in the sample.

### Sample

The methods and systems described herein can be used to analyze various types of samples.

In some embodiments, the method comprises acquiring a sample. In some embodiments, the sample comprises a physiological fluid. In some embodiments, the physiological fluid is plasma. In some embodiments, the physiological fluid is serum. In some embodiments, the physiological fluid is blood. In some embodiments, the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit.

In some embodiments, the sample or physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9. In some embodiments, the sample or physiological fluid has a pH of serum or plasma (e.g., 7.3-7.5). In some embodiments, the sample or physiological fluid has a pH of a tumor microenvironment (e.g., pH 5.6 to 6.8).

In some embodiments, the sample is diluted after being obtained from the subject. In some embodiments, the sample is not diluted after being obtained from the subject.

In some embodiments, the sample is not acquired from a subject, e.g., a physiological fluid reconstituted with the LNP. In some embodiments, the sample is acquired from a subject. In some embodiments, the subject is a healthy subject. In some embodiments, the subject has, or is likely to have, a disorder, e.g., an infection, a cancer, or an autoimmune disorder. In some embodiments, the subject is a human. In some embodiments, the subject is an animal (e.g., a non-human animal), e.g., a mammal, e.g., a mouse, a rat, or a primate.

### ENUMERATED EMBODIMENTS

1. A method of analyzing a lipid nanoparticle (LNP), comprising:
(optional) acquiring a sample comprising the LNP (e.g., in a physiological fluid);
subjecting the sample to a size-exclusion chromatography (SEC); and
acquiring a multi angle light scattering (MALS) signal from the sample,
thereby analyzing the LNP.
   2. The method of embodiment 1, further comprising:
subjecting a reference sample to the SEC; and
acquiring an MALS signal from the reference sample.
   3. The method of embodiment 2, further comprising comparing the MALS signal from the sample to the MALS signal from the reference sample.
   4. The method of any one of the preceding embodiments, further comprising subjecting the sample to LC-MS/MS, e.g., to determine (e.g., quantify) the component(s) of the LNP.
   5. The method of any one of the preceding embodiments, further comprising acquiring the UV absorbance (e.g., at λ=260 nm) for the sample, e.g., to determine (e.g., quantify) the amount of nucleic acid in the LNP.
   6. The method of any one of the preceding embodiments, wherein the method determines the stability of the LNP in the sample.
   7. The method of any one of the preceding embodiments, wherein the method determines the purity of the LNP in the sample.
   8. The method of any one of the preceding embodiments, wherein the method is suitable for monitoring manufacturing of the LNP.
   9. The method of any one of the preceding embodiments, wherein the method is suitable for determining the pharmacokinetics (PK) of the LNP.
   10. The method of any one of embodiments 1-9, wherein the sample is not obtained from a subject, e.g., a physiological fluid reconstituted with the LNP.
   11. The method of any one of embodiments 1-9, wherein the sample is acquired from a subject.
   12. The method of embodiment 10 or 11, wherein the subject is a healthy subject.
   13. The method of embodiment 10 or 11, wherein the subject has, or is likely to have, a disorder, e.g., an infection, a cancer, or an autoimmune disorder.
   14. The method of any one of embodiments 10-13, wherein the subject is a human.
   15. The method of any one of embodiments 10-13, wherein the subject is an animal, e.g., a mammal, e.g., a mouse, a rat, or a primate.
   16. A method of determining the stability of a lipid nanoparticle (LNP) in a subject, comprising:
(optionally) acquiring a first sample comprising the LNP from the subject;
subjecting the first sample to a size-exclusion chromatography (SEC);
acquiring a multi angle light scattering (MALS) signal from the first sample; (optionally) acquiring a second sample comprising the LNP from the subject;
subjecting the second sample to the SEC;
acquiring a second MALS signal from the second sample; and
comparing the first MALS signal and the second MALS signal,
wherein the comparison between the first MALS signal and the second MALS signal is indicative of the stability of the LNP in the subject,
thereby determining the stability of the LNP in the subject.
   17. The method of embodiment 16, wherein the subject has been administered with the LNP, e.g., at least 1, 6, 12, 18, or 24 hours, or 1, 2, 3, 4, 5, 6, or 7 days, before the first sample comprising the LNP is acquired.
   18. The method of embodiment 16 or 17, wherein the second sample is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first sample is acquired.
   19. The method of any one of embodiments 16-18, further comprising:
(optionally) acquiring a third sample comprising the LNP from the subject;
subjecting the third sample to the SEC;
acquiring a third MALS signal from the third sample; and
comparing the third MALS signal with the first MALS signal, the second MALS signal, or both,
wherein the comparison is indicative of the stability of the LNP in the subject.
   20. The method of embodiment 19, wherein the third sample is acquired after the second sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second sample is acquired.
   21. The method of embodiment 19 or 20, further comprising:
(optionally) acquiring a fourth sample comprising the LNP from the subject;
subjecting the fourth sample to the SEC;
acquiring a fourth MALS signal from the fourth sample; and
comparing the fourth MALS signal with one or more (e.g., all) of the first MALS signal, the second MALS signal, or the third MALS signal,
wherein the comparison is indicative of the stability of the LNP in the subject.
   22. The method of embodiment 21, wherein the fourth sample is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third sample is acquired.
   23. The method of embodiment 21 or 22, further comprising:
(optionally) acquiring a fifth sample comprising the LNP from the subject;
subjecting the fifth sample to the SEC;
acquiring a fifth MALS signal from the fifth sample; and
comparing the fourth MALS signal with one or more (e.g., all) of the first MALS signal, the second MALS signal, the third MALS signal, or the fourth MALS signal,
wherein the comparison is indicative of the stability of the LNP in the subject.
   24. The method of embodiment 23, wherein the fifth sample is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth sample is acquired.
   25. The method of any one of embodiments 16-24, wherein the LNP in the second or subsequent sample has a change to the LNP composition, e.g., lipid, nucleic acid, or both, compared to the LNP in the first or prior sample, optionally wherein the size (e.g., Rₕ) of the LNP in the second or subsequent sample is substantially identical to the size (e.g., Rₕ) of the LNP in the first or prior sample.
   26. The method of any one of embodiments 16-25, wherein the subject is a healthy subject.
   27. The method of any one of embodiments 16-25, wherein the subject has, or is likely to have, a disorder, e.g., an infection, a cancer, or an autoimmune disorder.
   28. The method of any one of embodiments 16-27, wherein the subject is a human.
   29. The method of any one of embodiments 16-27, wherein the subject is an animal, e.g., a mammal, e.g., a mouse, a rat, or a primate.
   30. A method of determining the stability of a lipid nanoparticle (LNP) in a sample comprising:
(optionally) acquiring a first aliquot of the sample;
subjecting the first aliquot to a size-exclusion chromatography (SEC);
acquiring a multi angle light scattering (MALS) signal from the first aliquot;
(optionally) acquiring a second aliquot of the sample;
subjecting the second sample to the SEC;
acquiring a second MALS signal from the second aliquot; and
comparing the first MALS signal and the second MALS signal,
wherein the comparison between the first MALS signal and the second MALS signal is indicative of the stability of the LNP in the sample,
thereby determining the stability of the LNP in the sample.
   31. The method of embodiment 30, wherein the second aliquot is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first aliquot is acquired.
   32. The method of embodiment 30 or 31, further comprising:
(optionally) acquiring a third aliquot of the sample;
subjecting the third sample to the SEC;
acquiring a third MALS signal from the third aliquot; and
comparing the third MALS signal with the first MALS signal, the second MALS signal, or both,
wherein the comparison is indicative of the stability of the LNP in the sample.
   33. The method of embodiment 32, wherein the third aliquot is acquired after the second aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second aliquot is acquired.
   34. The method of embodiment 32 or 33, further comprising:
(optionally) acquiring a fourth aliquot of the sample;
subjecting the fourth sample to the SEC;
acquiring a fourth MALS signal from the fourth aliquot; and
comparing the fourth MALS signal with one or more (e.g., all) of the first MALS signal, the second MALS signal, or the third MALS signal,
wherein the comparison is indicative of the stability of the LNP in the sample.
   35. The method of embodiment 34, wherein the fourth aliquot is acquired after the third aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third aliquot is acquired.
   36. The method of embodiment 34 or 35, further comprising:
(optionally) acquiring a fifth aliquot of the sample;
subjecting the fifth aliquot to the SEC;
acquiring a fifth MALS signal from the fifth aliquot; and
comparing the fourth MALS signal with one or more (e.g., all) of the first MALS signal, the second MALS signal, the third MALS signal, or the fourth MALS signal,
wherein the comparison is indicative of the stability of the LNP in the sample.
   37. The method of embodiment 36, wherein the fifth aliquot is acquired after the fourth aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth aliquot is acquired.
   38. The method of any one of embodiments 30-37, wherein the LNP in the second or subsequent aliquot has a change to the LNP composition, e.g., lipid, nucleic acid, or both, compared to the LNP in the first or prior aliquot, optionally wherein the size (e.g., Rₕ) of the LNP in the second or subsequent aliquot is substantially identical to the size (e.g., Rₕ) of the LNP in the first or prior aliquot.
   39. The method of any one of embodiments 30-38, wherein the sample is not acquired from a subject, e.g., a physiological fluid reconstituted with the LNP.
   40. The method of any one of embodiments 30-38, wherein the sample is acquired from a subject.
   41. The method of embodiment 39 or 40, wherein the subject is a healthy subject.
   42. The method of embodiment 39 or 40, wherein the subject has, or is likely to have, a disorder, e.g., an infection, a cancer, or an autoimmune disorder.
   43. The method of any one of embodiments 39-42, wherein the subject is a human.
   44. The method of any one of embodiments 39-42, wherein the subject is an animal, e.g., a mammal, e.g., a mouse, a rat, or a primate.
   45. A method of determining the purity of a lipid nanoparticle (LNP) in a sample, comprising:
(optionally) acquiring a first aliquot of the sample;
subjecting the first aliquot to a size-exclusion chromatography (SEC);
acquiring a multi angle light scattering (MALS) signal from the first aliquot;
(optionally) acquiring a second aliquot of the sample;
subjecting the second aliquot to the SEC;
acquiring a second MALS signal from the second aliquot; and
comparing the first MALS signal and the second MALS signal to determine the stability of the LNP in the sample, which is indicative of the purity of the LNP in the sample,
thereby determining the purity of the LNP in the sample.
   46. The method of embodiment 45, wherein the second aliquot is acquired after the first aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first aliquot is acquired.
   47. The method of embodiment 45 or 46, further comprising:
(optionally) acquiring a first reference aliquot of a reference sample comprising an LNP;
subjecting the first aliquot to the SEC;
acquiring an MALS signal from the first reference aliquot;
(optionally) acquiring a second reference aliquot of the reference sample;
subjecting the second reference aliquot to the SEC;
acquiring a second MALS signal from the second reference aliquot; and
comparing the first MALS signal and the second MALS signal to determine the stability of the LNP in the reference sample.
   48. The method of any one of embodiments 45-47, further comprising comparing the stability of the LNP in the sample with the stability of the LNP in the reference sample, thereby determining the purity of the LNP in the sample.
   49. The method of any one of embodiments 45-48, further comprising:
(optionally) acquiring a third aliquot of the sample (or the reference sample);
subjecting the third aliquot to the SEC;
acquiring a third MALS signal from the third aliquot; and
comparing the third MALS signal with the first MALS signal, the second MALS signal, or both.
   50. The method of embodiment 49, wherein the third aliquot is acquired after the second aliquot is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second aliquot is acquired.
   51. The method of embodiment 49 or 50, further comprising:
(optionally) acquiring a fourth aliquot of the sample (or the reference sample);
subjecting the fourth aliquot to the SEC;
acquiring a fourth MALS signal from the fourth aliquot; and
comparing the fourth MALS signal with one or more (e.g., all) of the first MALS signal, the second MALS signal, or the third MALS signal.
   52. The method of embodiment 51, wherein the fourth aliquot is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third aliquot is acquired.
   53. The method of embodiment 51 or 52, further comprising:
(optionally) acquiring a fifth aliquot of the sample (or the reference sample);
subjecting the fifth sample to the SEC;
acquiring a fifth MALS signal from the fifth aliquot; and
comparing the fourth MALS signal with one or more (e.g., all) of the first MALS signal, the second MALS signal, the third MALS signal, or the fourth MALS signal.
   54. The method of embodiment 53, wherein the fifth aliquot is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth aliquot is acquired.
   55. The method of any one of embodiments 45-54, wherein the LNP in the sample has an impurity due to incompletely de-protected form of the LNP.
   56. The method of any one of embodiments 45-55, wherein the sample is not acquired from a subject, e.g., a physiological fluid reconstituted with the LNP.
   57. The method of any one of embodiments 45-55, wherein the sample is acquired from a subject.
   58. The method of embodiment 56 or 57, wherein the subject is a healthy subject.
   59. The method of embodiment 56 or 57, wherein the subject has, or is likely to have, a disorder, e.g., an infection, a cancer, or an autoimmune disorder.
   60. The method of any one of embodiments 56-59, wherein the subject is a human.
   61. The method of any one of embodiments 56-59, wherein the subject is an animal, e.g., a mammal, e.g., a mouse, a rat, or a primate.
   62. A method of monitoring a process of manufacturing a lipid nanoparticle (LNP), comprising:
(optionally) acquiring a first sample comprising an LNP from the process of manufacturing the LNP;
subjecting the first sample to a size-exclusion chromatography (SEC); and
acquiring a multi angle light scattering (MALS) signal from the first sample,
thereby monitoring the process of manufacturing the LNP.
   63. The method of embodiment 62, further comprising:
(optionally) acquiring a second sample comprising the LNP from the process of manufacturing the LNP;
subjecting the second sample to the SEC; and
acquiring a second MALS signal from the second sample.
   64. The method of embodiment 63, wherein the second sample is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first sample is acquired.
   65. The method of embodiment 63 or 64, further comprising:
(optionally) acquiring a third sample comprising the LNP from the process of manufacturing the LNP;
subjecting the third sample to the SEC; and
acquiring a third MALS signal from the third sample.
   66. The method of embodiment 65, wherein the third sample is acquired after the second sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second sample is acquired.
   67. The method of embodiment 65 or 66, further comprising:
(optionally) acquiring a fourth sample comprising the LNP from the process of manufacturing the LNP;
subjecting the fourth sample to the SEC; and
acquiring a fourth MALS signal from the fourth sample.
   68. The method of embodiment 67, wherein the fourth sample is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third sample is acquired.
   69. The method of embodiment 67 or 68, further comprising:
(optionally) acquiring a fifth sample comprising the LNP from the process of manufacturing the LNP;
subjecting the fifth sample to the SEC; and
acquiring a fifth MALS signal from the fifth sample.
   70. The method of embodiment 69, wherein the fifth sample is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth sample is acquired.
   71. A method of determining the pharmacokinetics (PK) of a lipid nanoparticle (LNP), comprising:
(optionally) acquiring a first sample comprising an LNP from a subject;
subjecting the first sample to a size-exclusion chromatography (SEC); and
acquiring a multi angle light scattering (MALS) signal from the first sample,
thereby determining the PK of the LNP.
   72. The method of embodiment 71, further comprising:
(optionally) acquiring a second sample comprising the LNP from the subject;
subjecting the second sample to the SEC; and
acquiring a second MALS signal from the second sample.
   73. The method of embodiment 72, wherein the second sample is acquired after the first sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the first sample is acquired.
   74. The method of embodiment 72 or 73, further comprising:
(optionally) acquiring a third sample comprising the LNP from the subject;
subjecting the third sample to the SEC; and
acquiring a third MALS signal from the third sample.
   75. The method of embodiment 74, wherein the third sample is acquired after the second sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the second sample is acquired.
   76. The method of embodiment 73 or 74, further comprising:
(optionally) acquiring a fourth sample comprising the LNP from the subject;
subjecting the fourth sample to the SEC; and
acquiring a fourth MALS signal from the fourth sample.
   77. The method of embodiment 76, wherein the fourth sample is acquired after the third sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the third sample is acquired.
   78. The method of embodiment 76 or 77, further comprising:
(optionally) acquiring a fifth sample comprising the LNP from the subject;
subjecting the fifth sample to the SEC; and
acquiring a fifth MALS signal from the fifth sample.
   79. The method of embodiment 78, wherein the fifth sample is acquired after the fourth sample is acquired, e.g., at least 15, 30, 45, 60, 75, 90, 105, or 120 minutes, 1, 2, 3, 4, 5, 6, 7, 8, 9, 10, 11, 12, 13, 14, 15, 16, 17, 18, 19, 20, 21, 22, 23, 24, 36, or 48 hours, after the fourth sample is acquired.
   80. The method of any of embodiments 71-79, wherein the subject is a healthy subject.
   81. The method of any of embodiments 71-79, wherein the subject has, or is likely to have, a disorder, e.g., an infection, a cancer, or an autoimmune disorder.
   82. The method of any one of embodiments 71-81, wherein the subject is a human.
   83. The method of any one of embodiments 71-81, wherein the subject is an animal, e.g., a mammal, e.g., a mouse, a rat, or a primate.
   84. The method of any one of embodiments 1-83, wherein the SEC is performed in a single-column configuration.
   85. The method of any one of embodiments 1-83, wherein the SEC is performed in a dual-column configuration, e.g., using two columns with different pore sizes, e.g., a first column with a first average pore size and a second column with a second average pore size.
   86. The method of embodiment 85, wherein the first average pore size is greater than the second average pore size.
   87. The method of embodiment 85 or 86, wherein the first average pore size has a molecular weight cut-off (MWCO) of about 1 MDa or more, e.g., about 2 MDa or more, 5 MDa or more, 10 MDa or more, 15 MDa or more, or 20 MDa or more.
   88. The method of any one of embodiments 85-87, wherein the first average pore size has an MWCO of about 20 MDa or less, e.g., about 15 MDa or less, 10 MDa or less, 5 MDa or less, 2 MDa or less, or 1 MDa or less.
   89. The method of any one of embodiments 85-88, wherein the first average pore size has an MWCO of about 1 MDa to 20 MDa, e.g., about 2 MDa to 15 MDa, 5 MDa to 10 MDa, 1 MDa to 15 MDa, 1 MDa to 10 MDa, 1 MDa to 5 MDa, 1 MDa to 2 MDa, 15 MDa to 20 MDa, 10 MDa to 20 MDa, 5 MDa to 20 MDa, 2 MDa to 20 MDa, 2 MDa to 10 MDa, 5 MDa to 15 MDa, or 8 MDa to 12 MDa, e.g., about 10 MDa.
   90. The method of any one of embodiments 85-89, wherein the second average pore size has an MWCO of about 10 MDa or less, e.g., about 5 MDa or less, 2 MDa or less, 1 MDa or less, 0.5 MDa or less, 0.2 MDa or less, or 0.1 MDa or less.
   91. The method of any one of embodiments 85-90, wherein the second average pore size has an MWCO of about 0.1 MDa or more, e.g., about 0.2 MDa or more, 0.5 MDa or more, 1 MDa or more, 2 MDa or more, 5 MDa or more, or 10 MDa or more.
   92. The method of any one of embodiments 85-91, wherein the second average pore size has an MWCO of about 0.1 MDa to 10 MDa, e.g., about 0.2 MDa to 5 MDa, 0.5 MDa to 2 MDa, 0.1 MDa to 5 MDa, 0.1 MDa to 2 MDa, 0.1 MDa to 1 MDa, 0.1 MDa to 0.5 MDa, 0.1 MDa to 0.2 MDa, 5 MDa to 10 MDa, 2 MDa to 10 MDa, 1 MDa to 10 MDa, 0.5 MDa to 10 MDa, 0.2 MDa to 10 MDa, 0.2 MDa to 1 MDa, 1 MDa to 5 MDa, or 0.3 MDa to 0.7 MDa, e.g., about 0.5 MDa.
   93. The method of any one of embodiments 1-92, wherein the SEC is performed using a column having a length of 100 mm or more, e.g., about 150 mm or more, 200 mm or more, 250 mm or more, 300 mm or more, 400 mm or more, 500 mm or more, 600 mm or more, 700 mm or more, 800 mm or more, 900 mm or more, or 1000 mm or more, e.g., about 100 mm to 1000 mm, 150 mm to 500 mm, or about 200 mm to 300 mm, e.g., about 250 mm.
   94. The method of any one of embodiments 1-93, wherein the SEC is performed using a polymer-based column.
   95. The method of any one of embodiments 1-93, wherein the SEC is not performed using a silica-based column.
   96. The method of any one of embodiments 1-95, wherein the SEC is performed under an aqueous condition, e.g., with a bio-compatible buffer system, e.g., a phosphate buffer, a bicarbonate buffer, an acetate buffer, a Tris buffer, or any of the Good's buffers (e.g., as described in Good et al. Biochemistry. 1966; 5(2):467-77; Good and Izawa Methods Enzymol. 1972; 24:53-68: Ferguson et al. Anal Biochem. 1980 May; 104(2):300-10), e.g., any of MES. Bis-tris methane, ADA, Bis-tris propane, PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS, or a combination thereof.
   97. The method of any one of embodiments 1-96, wherein the SEC is not performed using an organic solvent (e.g., THF) as a mobile phase.
   98. The method of any one of embodiments 1-97, wherein the MALS is performed at a scattering angle of 150 and 90°.
   99. The method of any one of embodiments 1-98, wherein the MALS is performed using a laser wavelength of about 500 nm to 800 nm, e.g., about 600 nm to 700 nm, 500 nm to 700 mu, 600 nm to 800 nm, 500 nm to 600 nm, 700 nm to 800 nm, e.g., about 658 nm.
   100. The method of any one of embodiments 1-99, wherein the MALS is performed using a sample cell volume of about 1 μL to 50 μL, e.g., about 2 μL to 25 μL, 5 μL to 20 μL, 1 μL to 40 μL, 1 μL to 30 μL, 1 μL to 20 μL, 1 μL to 10 μL, 40 μL to 50 μL, 30 μL to 50 μL, 20 μL to 50 μL, 10 μL to 50 μL, 5 μL to 15 μL, or 10 μL to 20 μL, e.g., about 10 μL.
   101. The method of any one of embodiments 1-100, wherein the MALS is performed using a scattering volume of about 0.001 μL to 0.1 μL, e.g., about 0.005 μL to 0.05 μL, 0.001 μL to 0.05 μL, 0.001 μL to 0.01 μL, 0.05 μL to 0.1 μL, 0.01 μL to 0.1 μL, 0.005 μL to 0.1 μL, or 0.005 μL to 0.05 μL, e.g., about 0.01 μL.
   102. The method of any one of embodiments 1-101, wherein the MALS is performed at a temperature range of about 20° C. to 70° C., e.g., about 25° C. to 75° C. or 30° C. to 60° C.
   103. The method of any one of embodiments 1-102, wherein the MALS is performed with a temperature stability of no more than f 1° C., e.g., no more than ±0.5° C. or ±0.2° C.
   104. The method of any one of embodiments 1-103, wherein the MALS is performed at a pH range of about 1-12, e.g., about 2-11 or 2-10.
   105. The method of any one of embodiments 1-104, wherein the size of the LNP is determined, e.g., the hydrodynamic radius (Rₕ) of the LNP is determined.
   106. The method of any one of embodiments 1-105, wherein the molecular weight (MW) of the LNP is determined, e.g., one or more (e.g., 2, 3, 4, or 5) of the weight average MW (M_{w}), the number average MW (Mₙ), the MW corresponding to the maximum of the chromatographic peak (Mₚ), the z-average MW (M_{z} or M_{z+1}), or the viscosity average MW (Mᵥ), is determined.
   107. The method of any one of embodiments 1-106, wherein the polydispersity index is determined, e.g., by calculating the ratio of M_{w} to Mₙ.
   108. The method of any one of embodiments 1-107, wherein the R_{gw} of the LNP is determined, e.g., using a Zimm or partial Zimm approach.
   109. The method of any one of embodiments 1-108, wherein the half-life of the LNP is determined.
   110. The method of any one of embodiments 1-109, comprising comparing the acquired MALS signals directly or indirectly.
   111. The method of any one of embodiments 1-110, wherein a plurality of LNPs in the sample is analyzed.
   112. The method of any one of embodiments 1-111, wherein the method does not comprise a step of recovering the LNP, e.g., by ultracentrifugation.
   113. The method of any one of embodiments 1-112, wherein the method does not comprise a step of diluting the sample, e.g., to remove a high molecular weight component (e.g., plasma or serum component) that interferes with detection (e.g., detection by DLS).
   114. The method of any one of embodiments 1-113, wherein the method does not comprises a step of labeling the LNP, e.g., with a fluorophore.
   115. The method of any one of embodiments 1-114, wherein the method quantitatively detects the LNP in the sample.
   116. The method of any one of embodiments 1-115, wherein the LNP is loaded with a nucleic acid.
   117. The method of any one of embodiments 1-115, wherein the LNP is not loaded with a nucleic acid.
   118. The method of embodiment 116 or 117, wherein the nucleic acid is a therapeutic nucleic acid (TNA).
   119. The method of any one of embodiments 116-118, wherein the nucleic acid is an mRNA.
   120. The method of any one of embodiments 116-119, wherein the nucleic acid is a vaccine.
   121. The method of any one of embodiments 116-120, wherein the nucleic acid is a non-coding RNA, e.g., a small non-coding RNA.
   122. The method of any one of embodiments 116-121, wherein the nucleic acid is a small interfering RNA (siRNA), an antisense oligonucleotide (ASO), or a microRNA (miRNA).
   123. The method of any one of embodiments 116-118, wherein the nucleic acid is a DNA.
   124. The method of any one of embodiments 116-118, wherein the nucleic acid is an aptamer.
   125. The method of any one of embodiments 116-124, wherein the nucleic acid comprises one or more modified nucleotides.
   126. The method of any one of embodiments 116-125, wherein the nucleic acid is single stranded.
   127. The method of any one of embodiments 116-125, wherein the nucleic acid is double stranded.
   128. The method of any one of embodiments 1-127, wherein the sample comprises a physiological fluid.
   129. The method of embodiment 128, wherein the physiological fluid is plasma.
   130. The method of embodiment 128, wherein the physiological fluid is serum.
   131. The method of embodiment 128, wherein the physiological fluid is blood.
   132. The method of embodiment 128, wherein the physiological fluid is amniotic fluid, aqueous humor, bile, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, or vomit.
   133. The method of any of embodiments 128-132, wherein the physiological fluid has a pH of 2-10, e.g., a pH of 3-9, 4-8, 5-7, 2-8, 2-6, 2-4, 8-10, 6-10, 4-10, 2-10, 2-4, 3-5, 4-6, 6-8, or 7-9.
   134. The method of embodiment 133, wherein the physiological fluid has a pH of serum or plasma (e.g., 7.3-7.5).
   135. The method of embodiment 133, wherein the physiological fluid has a pH of a tumor microenvironment (e.g., pH 5.6 to 6.8).
   136. A system comprising a processor configured to perform the method of any one of embodiments 1-135.
   137. The system of embodiment 136, wherein the processor is configured to subject a sample or an aliquot of a sample to a size-exclusion chromatography (SEC).
   138. The system of embodiment 136 or 137, wherein the processor is further configured to obtain a multi angle light scattering (MALS) signal from the sample or the aliquot of the sample.

### EXAMPLES

### Example 1. Quantitative Analysis of Lipid Nanoparticle Stability in Physiological Fluids by Size-Exclusion Chromatography Coupled to Multi-Angle Light Scattering

### Introduction

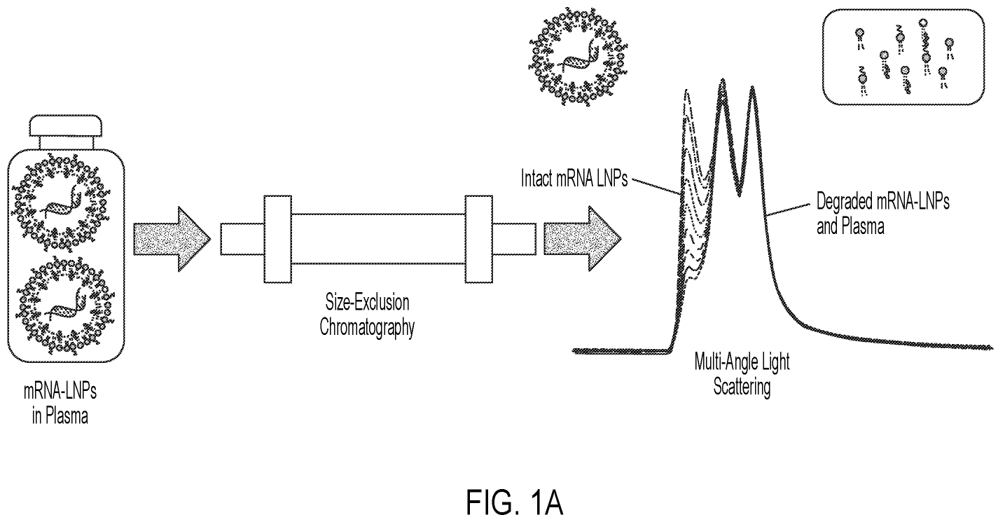

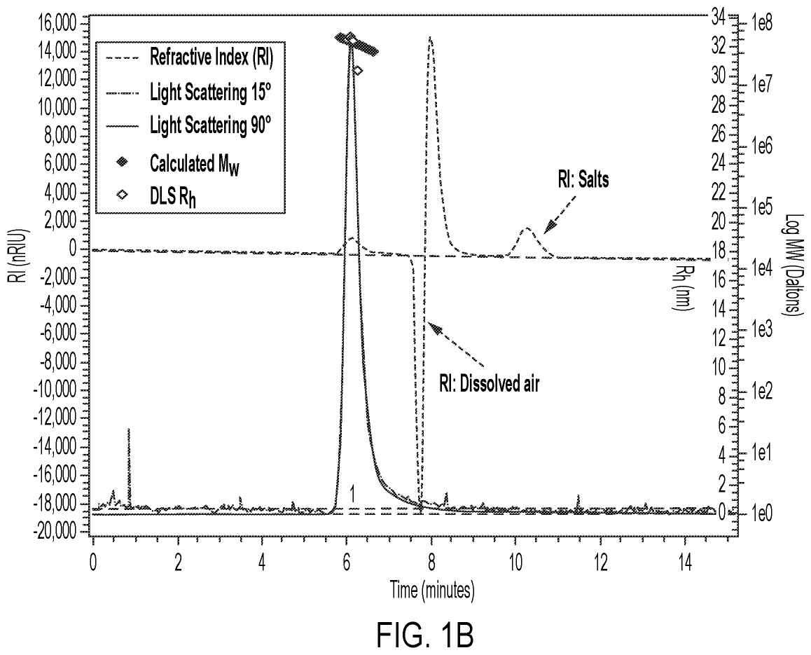

This Example demonstrates that stability of LNPs in undiluted plasma and serum can be measured accurately using size-exclusion chromatography coupled to multi-angle light scattering (SEC-MALS). An exemplary workflow for measurement of LNP stability is shown in FIG. 1A. The LNPs degraded with first-order kinetics in both plasma and serum with a half-life of approximately 85 min at room temperature. Changes in the apparent M_{w} and R_{g} detected by multi-angle light scattering indicated that LNP degradation in plasma was associated with a displacement of amino-, PEG- and helper lipids with proteins over time. In contrast, LNP compositions were unaffected by serum. Finally, LNP stability in plasma was compromised by trace impurities in lipid ingredients (about 1%) in this study. Taken together, these findings show that SEC-MALS is a useful tool for lipid formulation optimization and quality control.

### Methods

Human plasma powder (P9523-5ML), DDC Mass Spect Gold Serum (MSG3000-100ML), and human serum albumin (A3782-5G) were purchased from Sigma Aldrich.

Production of mRNA-LNPs—Lipid nanoparticle components ALC-0315, ALC-0159, DSPC and cholesterol were purchased from MedhemExpress (HY-138170, HY-138300, HY-W040193, and HY-N0322 respectively). mRNA was in-vitro transcribed from a 3800 nt PCR-amplified dsDNA template using T7 RNA polymerase (NEB, M0251L), purified Monarch RNA cleanup kit spin columns (NEB T2040L), quantitated by UV spectroscopy, then dissolved in 1 mM sodium acetate buffer (pH 4.8) to form the aqueous phase. mRNA-LNPs were produced using the same composition as Cominarty. ALC-0315, ALC-0159, DSPC and cholesterol were dissolved in ethanol at a molar ratio of 46.3:1.6:9.4:42.7 to form the organic phase. The organic phase and aqueous phase with volume ratio of 3:1 were allowed to undergo controlled mixing in a benchtop microfluidic device (NanoAssemblr Platform, Precision NanoSystems) at a flow rate of 12 mL/min. The N/P ratio was 6:1 for preparation of mRNA-LNPs. The resulting mRNA-LNPs were buffer exchanged into physiological saline solution and concentrated to a total lipid concentration of ˜2.2 mg/mL, then characterized by batch DLS (Zetasizer, Malvern Panalytical) or SEC-MALS. Empty LNPs were prepared in an identical manner, but without inclusion of mRNA.

mRNA-LNP incubation in human plasma, serum and albumin—Human plasma powder was reconstituted in MilliQ water according to the manufacturer's instructions, then centrifuged at 10,000×g for 10 mins and 0.2 μm filtered to remove particulates. Serum albumin was dissolved in 50 mM sodium phosphate buffer (pH 7.2) at a concentration of 40 mg/ml. mRNA-LNPs were diluted 1:10 (v/v) into filtered plasma, serum, or serum albumin solutions, then immediately injected at 15 min intervals for SEC-MALS analysis.

SEC-MALS—The following equipment stack was used: (i) Agilent 1290 Infinity II Bio LC with quaternary pump, (ii) Agilent 1260 Infinity II Bio-SEC Multi-Detector System. (iii) Agilent 1290 Infinity II Diode Array Detector, and (iv) Agilent 1290 Infinity II Refractive Index Detector. The diode array detector was used primarily for system calibration and was not required for light scattering experiments as the refractive index detector was used for concentration estimates. The quaternary pump was used in isocratic mode with only a single solvent line active to reduce baseline fluctuations in the refractive index detector. The Multi-Detector System was equipped to perform multi-angle light scattering at 15° and 90°, as well as dynamic light scattering. Two 4.6×250 mm, 8 μm, PL Aquagel-OH SEC columns with different pore sizes (PL1549-5801 and PL1549-5800) were used for this study. The mobile phase was triple-0.1 μm-filtered 50 mM sodium phosphate (pH 7.2) and was used at a flow rate of 0.6 ml/min.

Prior to sample analysis, both the Multi-Detector System and refractive index detector were allowed to equilibrate to 30° C. over 24 hours. SEC columns were flushed overnight at a low flow rate directly to waste, bypassing all detectors. About 1 hour prior to sample analysis, with the flow still bypassing all detectors, the flow rate was gradually increased to 0.6 ml/min. The SEC columns were then flushed at 0.6 ml/min for at least 5 mins before connecting the SEC columns to the detectors. The detectors were then flushed at 0.6 ml/min until all readings had reached low and stable baselines. Several 50 μl injections of 10 mg/ml BSA dissolved in mobile phase were used to ensure system suitability and to calibrate the system. Data acquisition and analysis were performed in Agilent Bio-SEC Software using a

$$\frac{dn}{dc}$$

value of 0.16 for mRNA-LNPs, and 0.185 for BSA.

LC-MS/MS quantification of mRNA-LNP lipid components—This assay was implemented on an Agilent 6545XT LC/Q-TOF, with an Agilent 1290 Infinity II Bio LC with binary pump as the front end.

For chromatography, a 2.1×50 mm, 1.9 μm InfinityLab Poroshell 120 phenyl hexyl column was used. The column was heated to 45° C. for analysis, and the flow rate set at 0.4 ml/min. Mobile phase A was 87% MeOH in 10 mM ammonium acetate, and mobile phase B was 90% ACN in 10 mM ammonium acetate. All solvents used were LC-MS grade. Prior to analysis, the column was first equilibrated at 0% mp B. During analysis, the gradient was set to 0% mp B for the first 0-2 min, after which mp B increased linearly to 100% from 2-7 min, followed by 3 min post time.

To generate calibration curves, a stock solution containing 2 mM ALC-0315, 2 mM ALC-0159, 2 mM DSPC and 20 mM Cholesterol was freshly prepared in methanol for each analysis. The calibration solution was then serially diluted in methanol to a minimum concentration of 0.1 nM ALC-0315, 0.1 nM ALC-0159, 0.1 nM DSPC and 1 nM Cholesterol. Prior to injecting samples, several injections of diluted calibration solution were performed to ensure consistent separation and mass spectrometric signal.

For LC-MS profiling of mRNA-LNP components the same chromatography method and mass spectrometer parameters were used, but the mass spectrometer was set to collect MS1 only.

Confirming the identity of the ALC-0315 impurity—Impurity fractions after reversed phase chromatography were collected and pooled in 2 ml snap-cap tubes, then evaporated to dryness in a centrifugal evaporator. Impurity samples were deprotected using 33% HBr/AcOH (0.5 ml) and trifluoroacetic acid (TFA, 2 ml) for 1 hr, then stored chilled at 4° C. prior to analysis. Control impurity samples were reconstituted in 87% MeOH for analysis without deprotection.

### Results

mRNA-LNPs and empty LNPs (without mRNA) were each diluted into 50 mM phosphate buffer (pH 7.2) to a final lipid concentration of 0.22 mg/ml before analysis with SEC-MALS to determine their physical characteristics by deconvolving refractive index and light scattering at 150 and 90° (LS15° and LS90°) chromatograms (FIG. 1B). SEC-MALS and batch DLS Rₕ measurements were in good agreement and indicated that both types of nanoparticles were essentially monodisperse (Table 1). Empty LNPs were smaller in size and had lower apparent M_{w} values than mRNA-LNPs, which was consistent with their lack of mRNA loading.

TABLE 1
Physical properties of mRNA-LNPs and Empty LNPs (N = 3)

| | | Rₕ | | | R_{gw} | M_{w} |
|---|---|---|---|---|---|---|
| | | (nm) | PD¹ | ζ (mV) | (nm) | (MDa) |
| mRNA- | Batch | 37.8 ± | 0.08 ± | −0.47 ± | — | — |
| LNPs | DLS | 0.15 | 0.01 | 0.82 | | |
| | SEC- | 35.7 ± | 1.03 ± | — | 28.8 ± | 42.5 ± |
| | MALS | 1.12 | 0.03 | | 2.3 | 2.3 |
| Empty | Batch | 35.3 ± | 0.08 ± | −0.56 ± | — | — |
| LNPs | DLS | 0.38 | 0.02 | 0.25 | | |
| | SEC- | 32.9 ± | 1.01 ± | — | 25.5 ± | 32.9 ± |
| | MALS | 1.0 | 0.01 | | 1.1 | 2.9 |

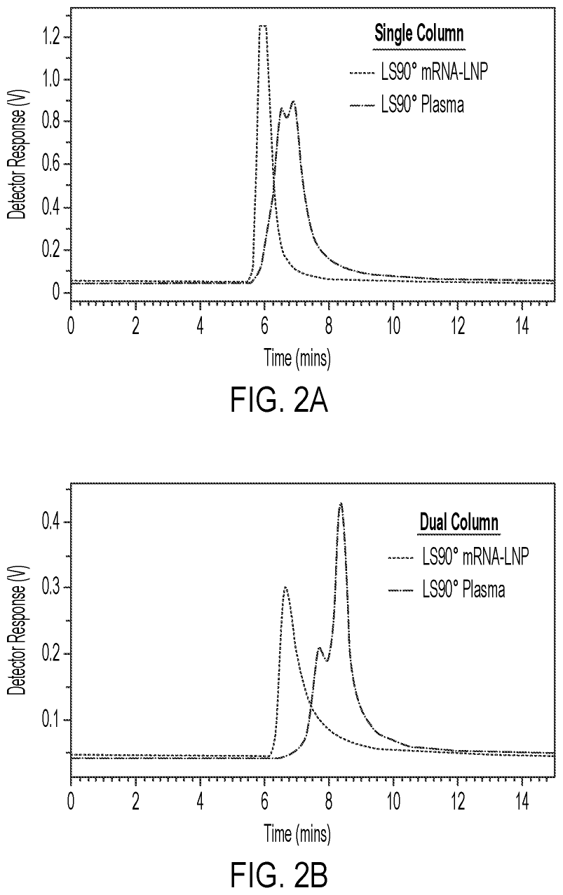

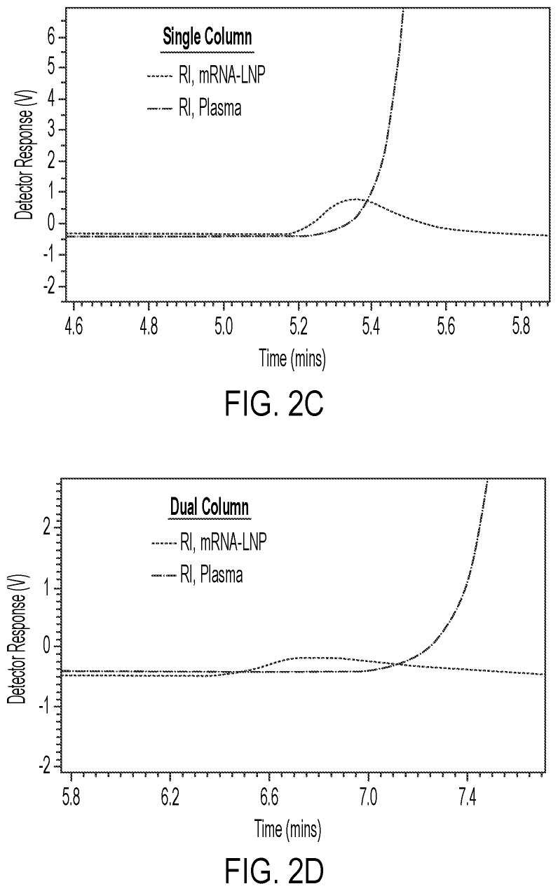

Separation in SEC results from transient inclusion of analytes into porous chromatography resins, whereas resolution depends on column length and average pore size. When a single column proves insufficient, columns of different average pore sizes (reflected by their molecular weight cutoffs, or MWCO) may be used to separate analytes over a wider range of molecular weights than can be addressed with a single column type. mRNA-LNPs and human plasma were injected separately in single- or dual-column configurations (FIGS. 2A-2D). The single-column configuration comprised a wide pore 4.6×250 mm PL aquagel-OH column (10 MDa MWCO), and the dual-column configuration comprised a wide pore column followed by a second narrower pore 4.6×250 mm aquagel-OH column (0.5 MDa MWCO) connected in series. The single-column configuration could not adequately resolve plasma components from mRNA-LNPs (FIG. 2A), whereas the dual-column configuration provided acceptable resolution for much of the mRNA-LNP peak (FIG. 2B). The refractive index signal from mRNA-LNPs was much smaller than plasma components, whereas co-elution would result in significant errors in mRNA-LNP concentration estimates, thereby causing inaccurate M_{w} calculations (FIGS. 2C-2D). Hence, good chromatographic separation was imperative for accurate analysis (FIG. 2D).

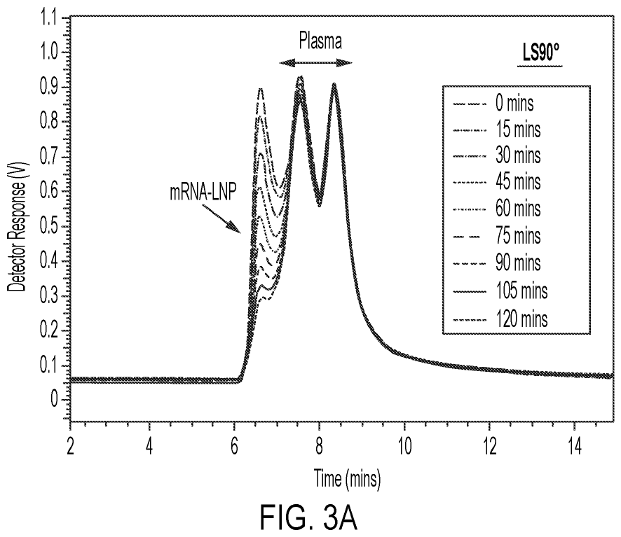

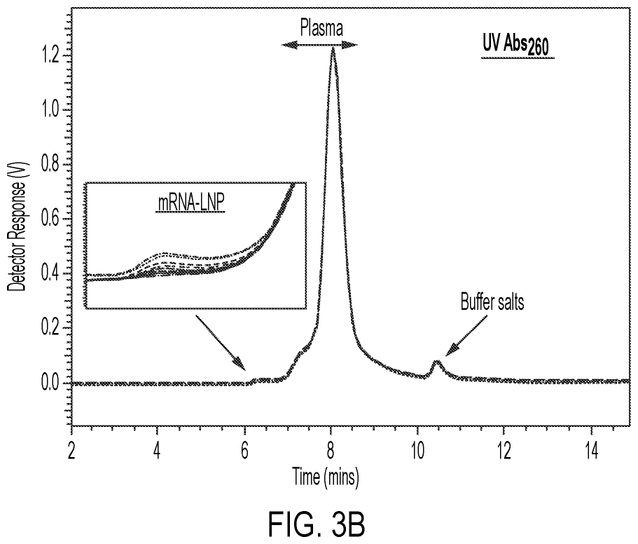

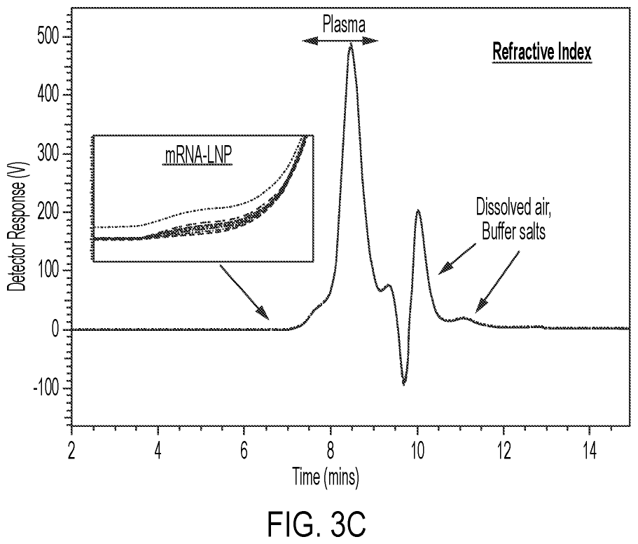

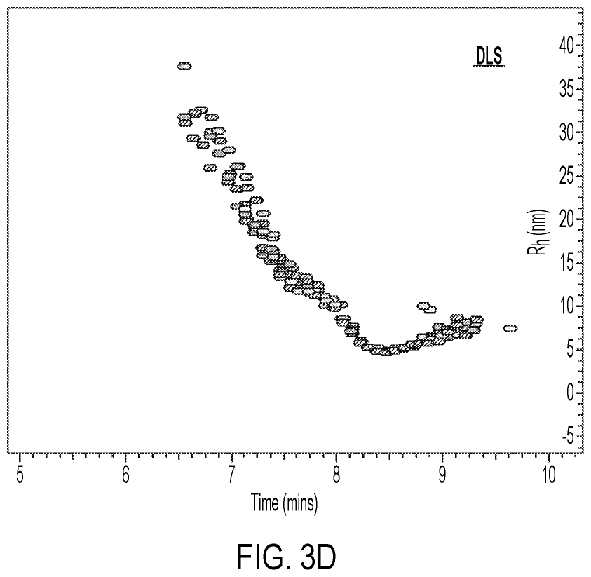

mRNA-LNPs were incubated in human plasma and injected at 15 min intervals for separation and analysis using dual-column SEC-MALS (FIG. 3A). The LS90° chromatograms clearly showed the mRNA-LNP peak at 6.7 min decreasing in intensity over time, indicating that mRNA-LNPs were degrading in human plasma. The plasma component peak at 7.7 min decreased slightly over time due to tailing from the mRNA-LNP peak. The plasma component peak at 8.2 min was baseline-separated from the mRNA-LNPs and showed no change in intensity, retention time or peak shape throughout the experiment, serving as an internal control for the quality of separation and consistency of injection volumes, UV absorbance at λ=260 nm can be used to detect the elution of encapsulated mRNA despite the lack of strong UV chromophores in nanoparticle lipids. FIGS. 3B-3C show overlaid UV absorbance and refractive index chromatograms for the same injection series. However, the observed signal was confounded by nanoparticles scattering incident light, making uncorrected UV absorbance an inaccurate means of estimating the concentration of mRNA-LNPs (FIG. 3B). As UV absorbance and refractive index detectors were far less sensitive to nanoparticles, they were less suitable than LS90° for accurately determining LNP degradation kinetics. Nevertheless, the decrease in mRNA-LNP peak intensities over time corroborated the LS90° result (FIG. 3C). DLS readings across all injections, which showed consistent separation of analytes by Rₕ from 6.5 to 8.4 min, confirmed that separation is primarily by a size exclusion mechanism (FIG. 3D).

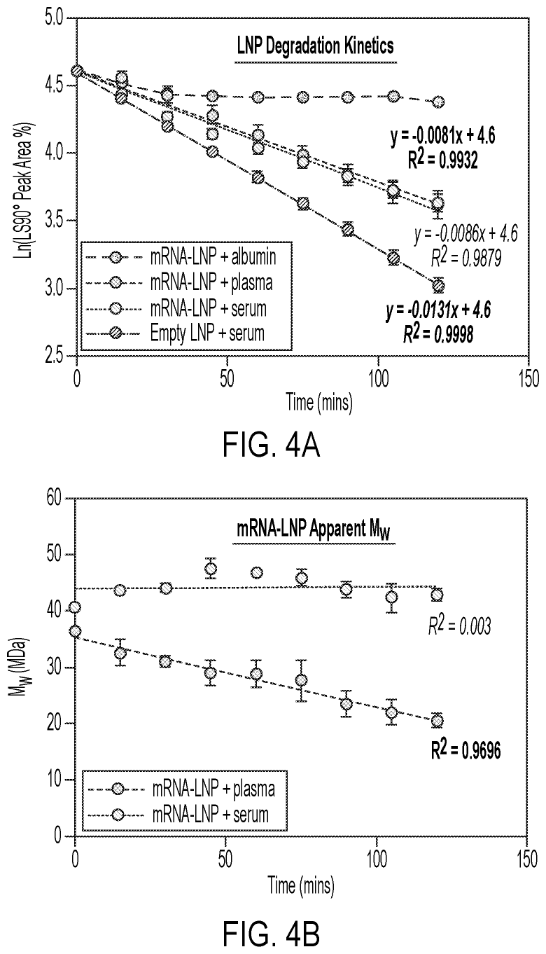

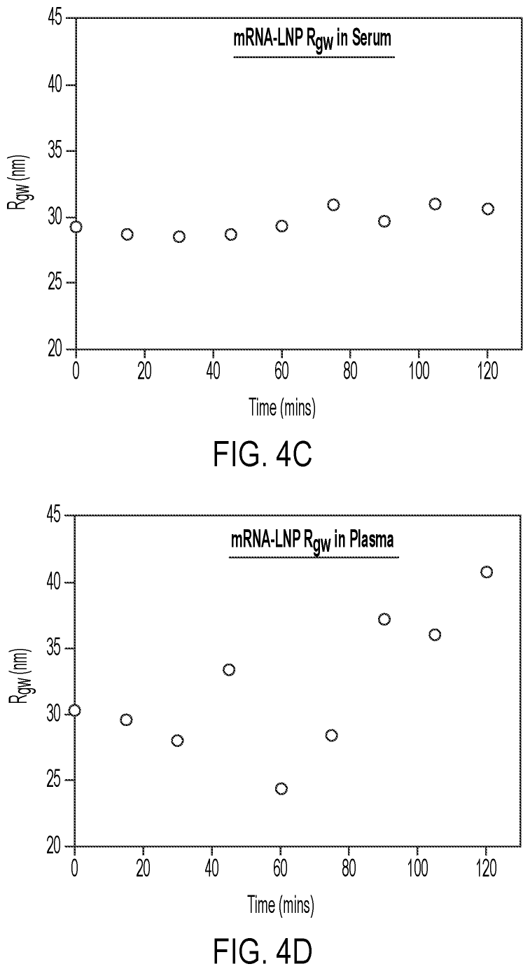

The degradation kinetics of mRNA-LNPs in human plasma and serum were averaged over three independent experiments (FIG. 4A). The degradation rate was well-described by first-order kinetics and was similar in both plasma and serum with a half-life between 80 and 85 min (blue and orange traces). In contrast, mRNA-LNPs were relatively stable in 40 mg/ml human serum albumin as peak areas do not change appreciably after 30 min. As mRNA-LNPs were stabilized by both hydrophobic interactions and coulombic electrostatic interactions between the negatively charged mRNA and positively charged amino-lipids, it was hypothesized that empty LNPs would degrade faster than mRNA-LNPs as negatively charged proteins in serum may interact with positively charged amino lipid, destabilizing the structure of LNPs. This was confirmed as the empty LNPs had a shorter half-life of 53 min in serum (green trace), thus demonstrating that the observed degradation rate was representative of LNP stability. Additionally, the apparent M_{w} of degrading mRNA-LNPs was averaged over three independent experiments (FIG. 4B). The apparent M_{w} of mRNA-LNPs remained unchanged at ˜43 MDa in serum but decreased significantly over time in plasma, indicating that the degradation process was different in the two environments. The decrease in apparent M_{w} in plasma occurred despite the Rₕ of degrading mRNA-LNPs remaining approximately the same, as indicated by the lack of change in retention times of the eluting mRNA-LNP peaks (FIG. 3A and FIG. 3D).

mRNA-LNP lipids have been progressively replaced by plasma proteins throughout the experiment, thereby changing the average composition of degrading mRNA-LNPs while maintaining their average Rₕ. As proteins have a higher average refractive index increment

$$\frac{dn}{dc}$$

of ˜0.19 compared to 0.16 for nanoparticle lipids, the average

$$\frac{dn}{dc}$$

of mRNA-LNPs degrading in plasma were hypothesized to increase over time in this scenario. The apparent M_{w} was inversely related to

$$\frac{dn}{dc}$$

as seen from the light scattering equation:

$${R}_{θ} {P}_{θ} = {M}_{w} K {( \frac{dn}{dc} )}^{2} C$$

where R_{θ} was the intensity of light scattering at angle θ, P_{θ} was the scattering function, M_{w} was the apparent molecular weight,

$$\frac{dn}{dc}$$

was the refractive index increment, C was the concentration, and K was a constant.

The R_{gw} of mRNA-LNPs degrading in serum remained approximately unchanged, whereas they increased over time in plasma (FIGS. 4C-4D). This observation indicated that heavier plasma proteins were replacing lipids on the periphery of mRNA-LNPs, causing an increase in the average radius of gyration. To confirm that mRNA-LNP compositions change in human plasma but not in serum, an LC-MS/MS method was used to quantify the molar ratio of Cholesterol:ALC-0315:DSPC:ALC-0159.

For mass spectrometry, three time segments were programmed: 0-1.3 min where flow was directed to waste, 1.3-4 min where flow was directed into the mass spectrometer with parameters optimized for Cholesterol analysis, and 4-7 mins where flow was directed into the mass spectrometer with parameters optimized for lipid analysis. The following parameters were used:

| Parameter | 0-1.3 min | 1.3-4 min | 4-7 min |
|---|---|---|---|
| Gas temp | 250 | 250 | 250 |
| Drying gas | 10 | 10 | 10 |
| Nebulizer | 35 | 60 | 35 |
| Sheath gas temp | 300 | 300 | 300 |
| Sheath gas flow | 12 | 12 | 12 |
| Vcap | 3500 | 3500 | 3500 |
| Nozzle voltage | 500 | 2000 | 500 |
| Fragmentor | 150 | 150 | 150 |
| Skimmer | 65 | 65 | 65 |
| Oct 1 RF Vpp | 750 | 750 | 750 |

Degrading mRNA-LNPs in plasma and serum were collected at 0 and 90 mins and their lipid components were quantified in three independent experiments.

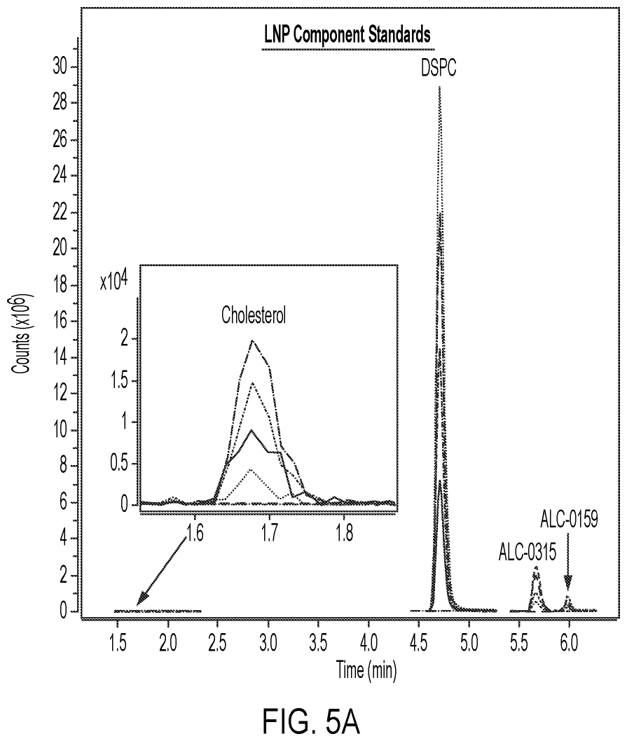

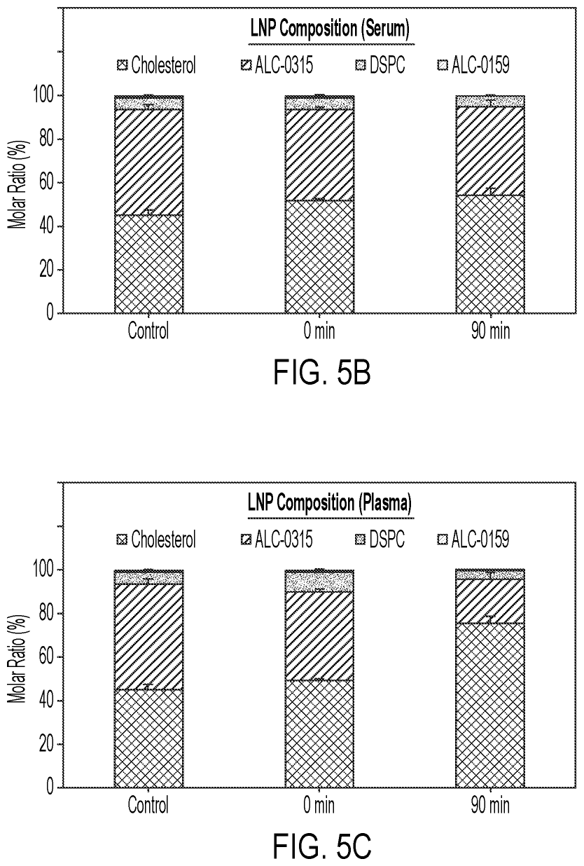

Parallel reaction monitoring (PRM) chromatograms were obtained for admixtures of LNP component standards injected at different quantities to demonstrate reproducibility of retention times and baseline separation of each component using a 7-minute separation method (FIG. 5A). In brief, parallel reaction monitoring was performed during the 1.3-4 min and 4-7 min time segments with the following precursor masses and collision energies:

| Time | | | | | | | |
|---|---|---|---|---|---|---|---|
| Segment | | Prec. m/z | Z | Ret Time | Delta RT | Iso width | CE |
| 1.3 | min | 369.65 | 1 | 1.8 | 1 | 4 m/z | 30 |
| 4 | min | 790.82 | 1 | 4.8 | 0.8 | 4 m/z | 50 |
| 4 | min | 766.91 | 1 | 5.7 | 0.4 | 4 m/z | 60 |
| 4 | min | 1184.10 | 2 | 6.1 | 0.4 | 4 m/z | 100 |

Control mRNA-LNPs that were exposed to neither serum nor plasma showed a Cholesterol:ALC-0315:DSPC:ALC-0159 molar ratio of 42.1:49.2:6.9:1.9 (FIGS. 5B-5C), similar to the theoretical molar ratio (42.7:46.3:9.4:1.6) used for the preparation of LNPs by microfluidics. At T=0 min, the proportion of ALC-0315, DSPC and ALC-0159 decreased slightly relative to Cholesterol in both serum and plasma (serum=51.7:41.7:5.3:1.2, plasma=49.2:40.8:8.4:1.5). At T=90 min, the LNP composition in serum showed little change in the major components Cholesterol and ALC-0315, but further decreases in DSPC and ALC-0159 were evident (54.4:40.7:4.5:0.4). However there was a significant decrease in the proportion of ALC-0315 in addition to decreases in DSPC and ALC-0159 (75.4:20.3:4.0:0.4) when incubated with plasma.

Taken together, both the light scattering and LC-MS/MS data indicated that plasma incubation resulted in significant changes to the mRNA-LNP composition during degradation, even in nanoparticles that retained their original Rₕ. Thus proteins such as clotting factors (e.g., fibrinogen), which are present in plasma but not in serum, appear to preferentially replace the amino-lipid ALC-0315 in mRNA-LNPs. Although the LC-MS/MS data showed that LNP compositions also changed during serum incubation, the changes occurred primarily through the displacement of the minor lipid components DSPC and ALC-0159, thus resulting in smaller overall changes to mRNA-LNP M_{w} and R_{gw}.

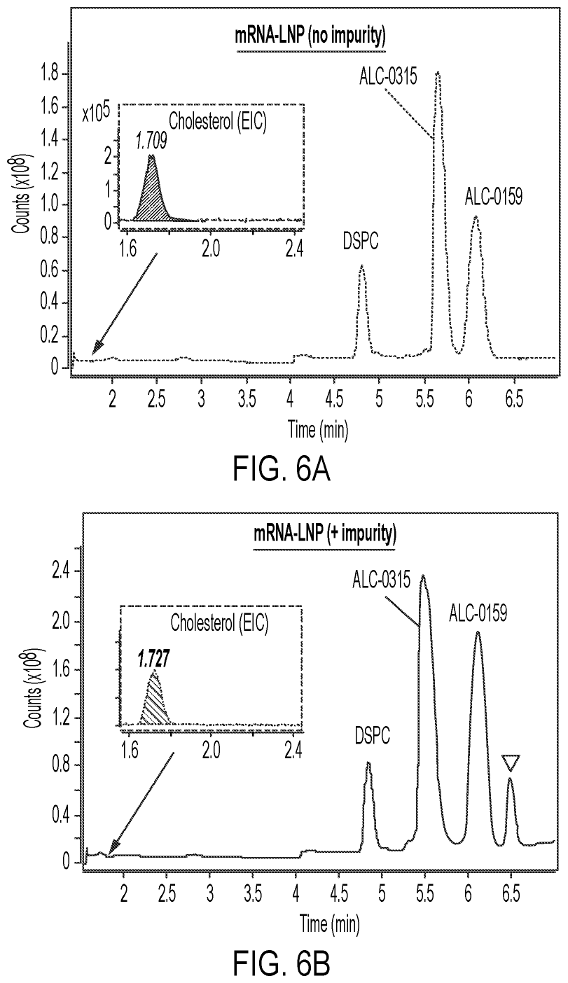

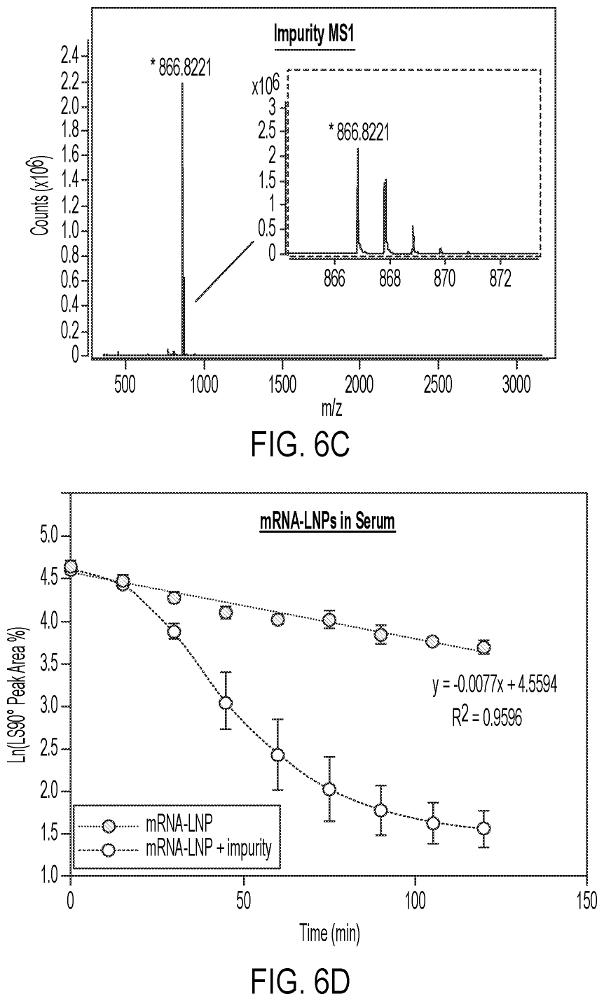

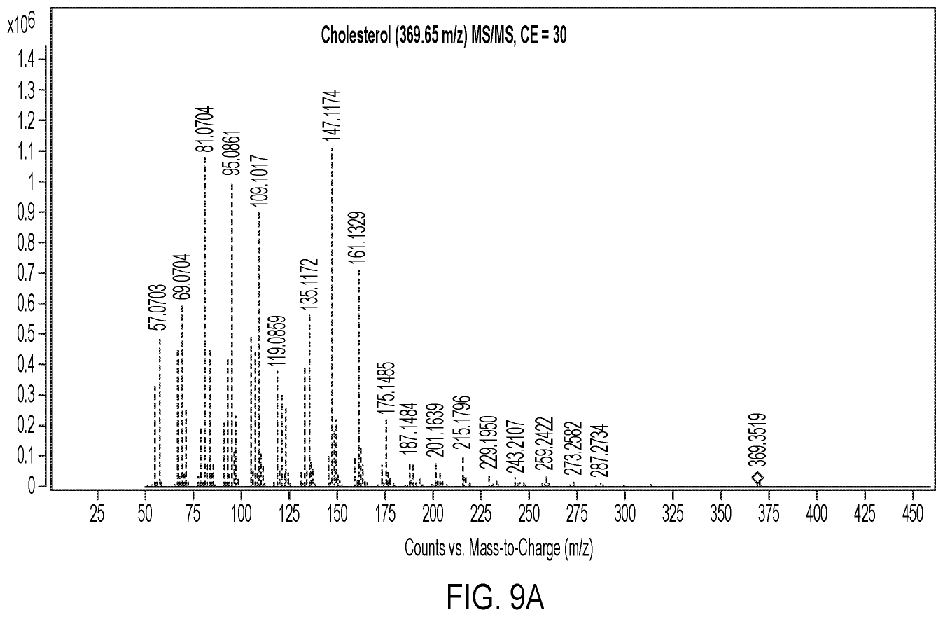

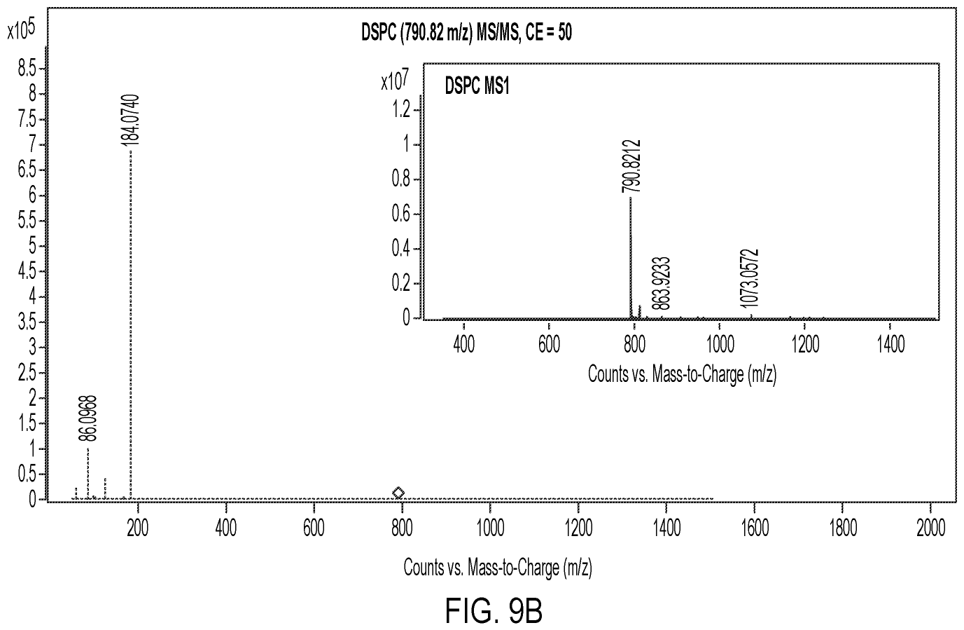

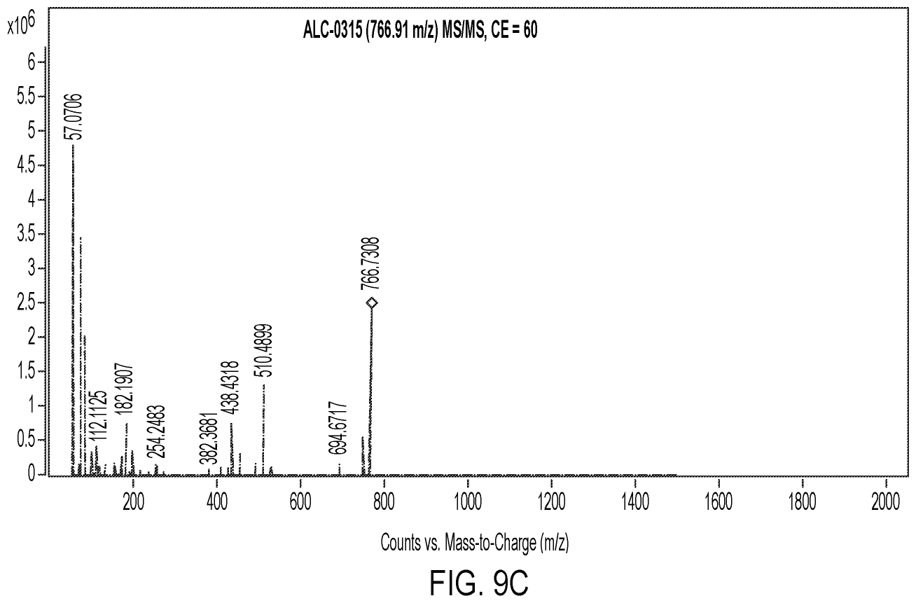

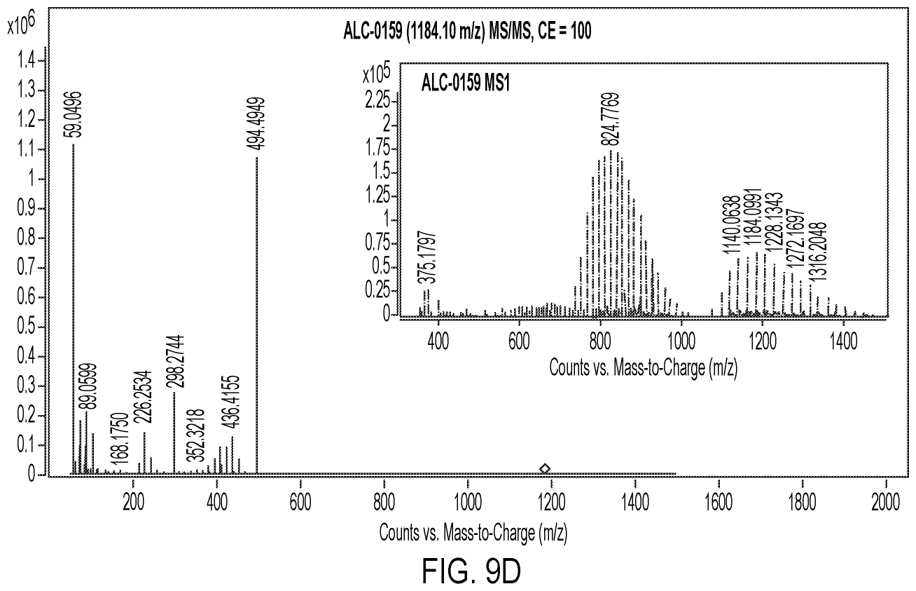

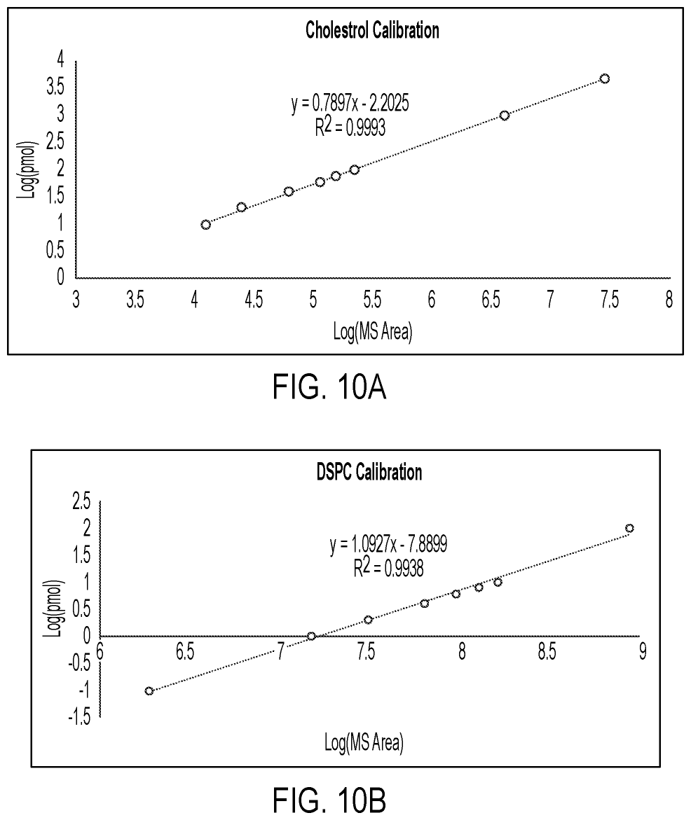

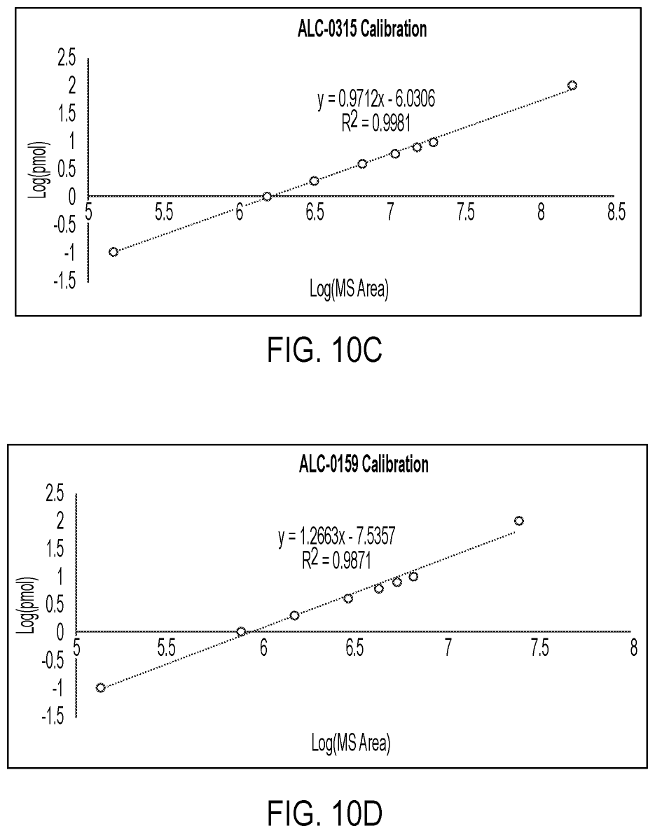

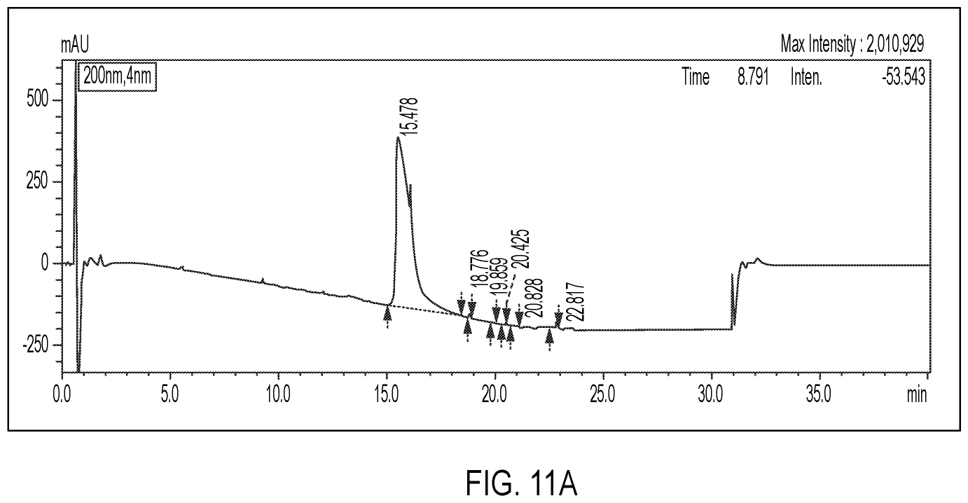

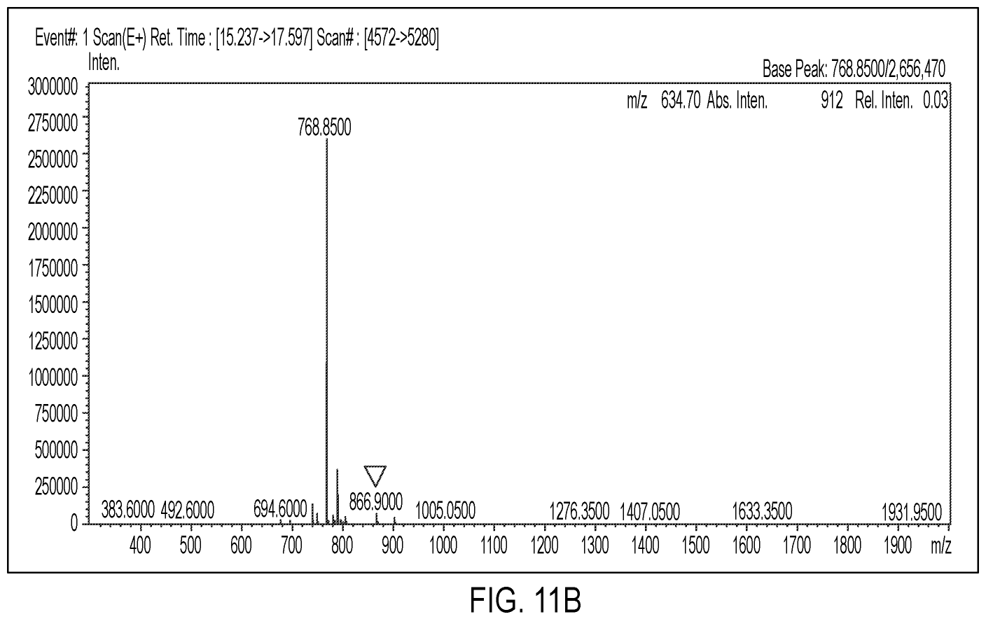

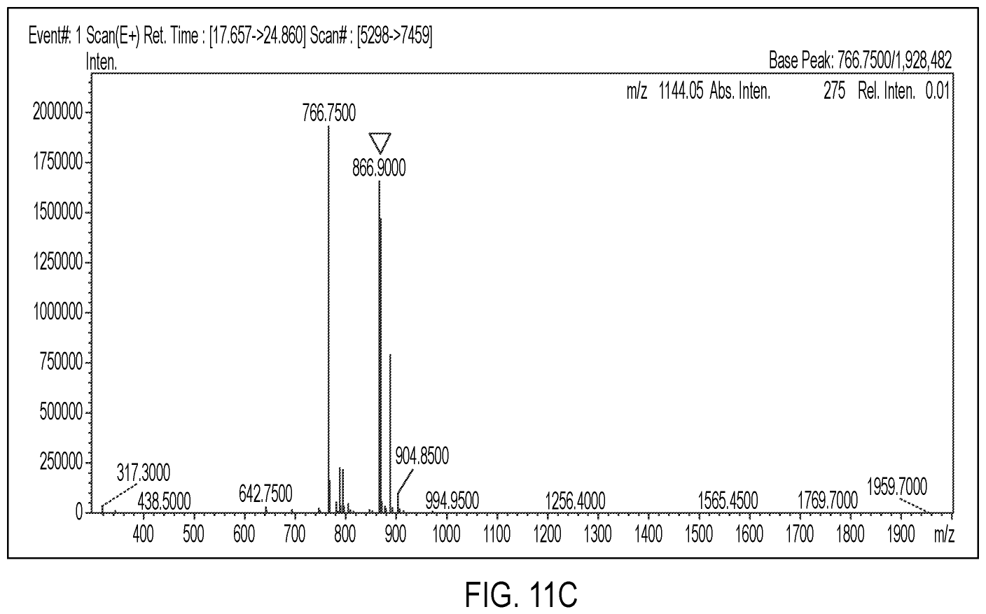

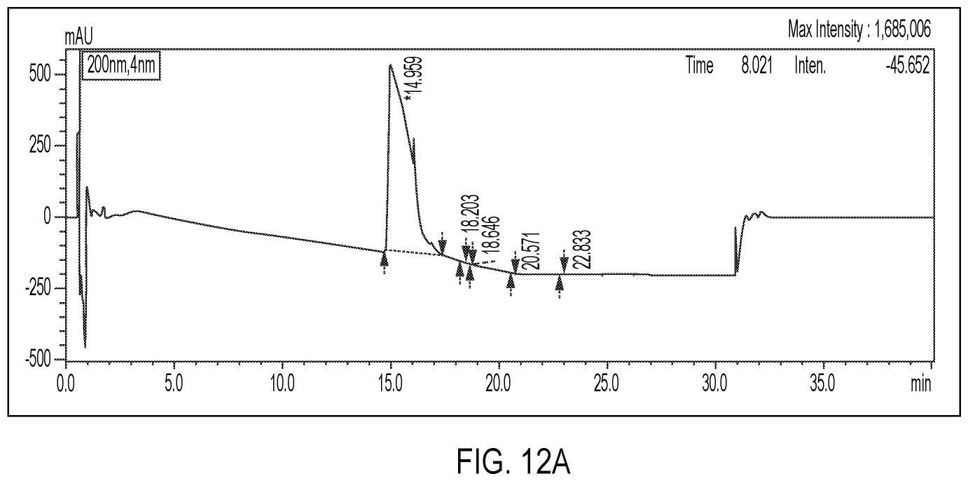

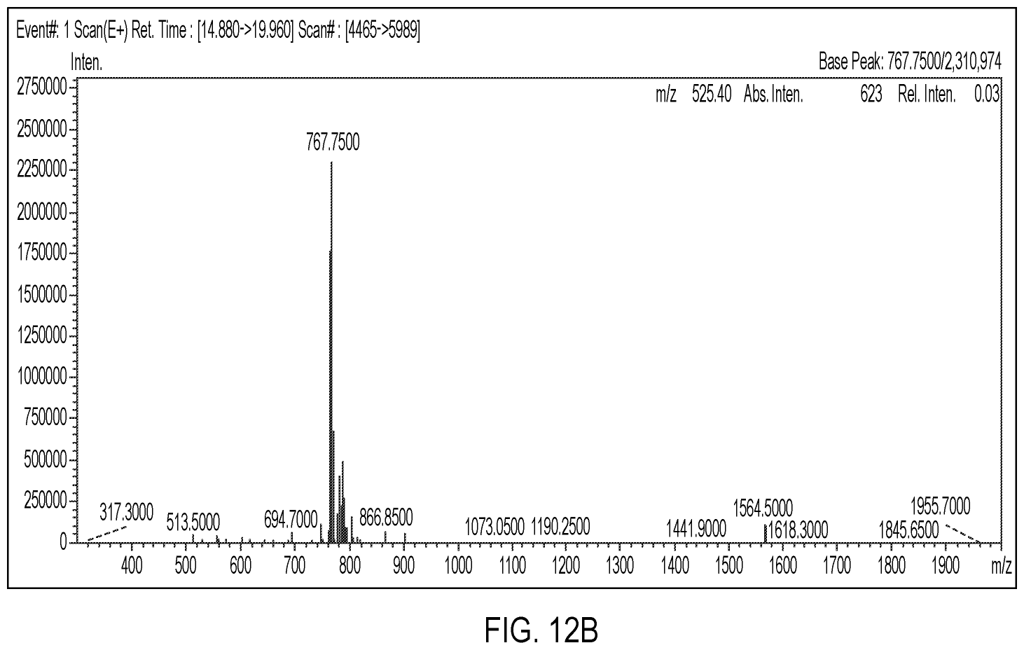

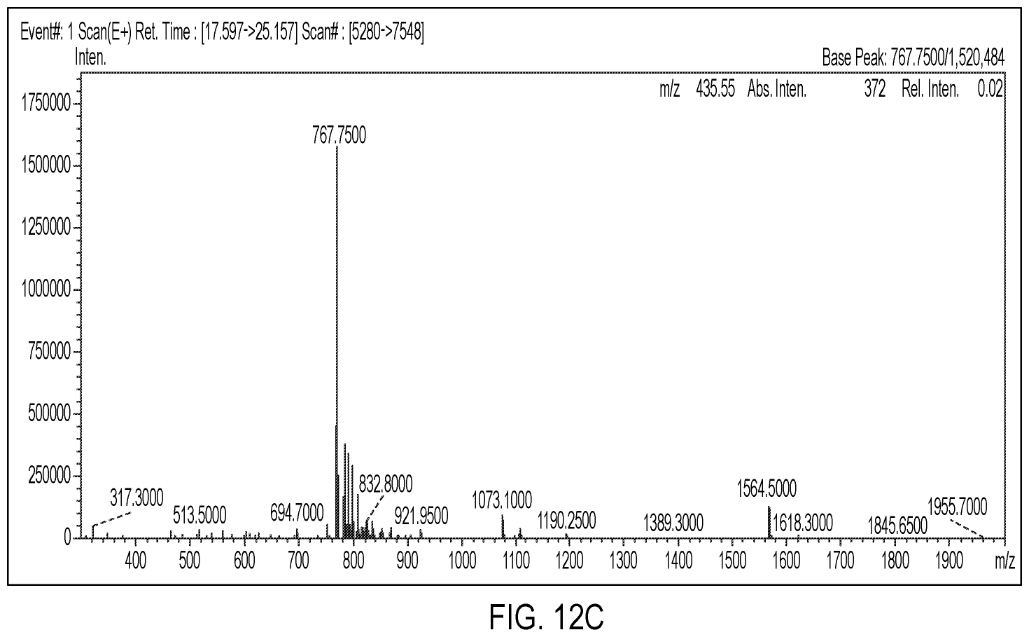

This SEC-MALS method was useful in assessing the impact of trace lipid impurities on the stability of mRNA-LNPs. Plasma component standards were identified via MS1 spectra (FIGS. 9A-9D). Calibration curves were generated for individual standards, as shown in FIGS. 10A-10D. Total ion chromatograms of the lipid components in two batches of mRNA-LNPs were made using different lot numbers of ALC-0315, but with the same lot numbers for all other components (FIGS. 6A-6B). In addition to the four expected standard components, the contaminated batch showed a fifth peak eluting at 6.5 min (FIG. 6B) with a monoisotopic mass of 866.8208 Da (FIG. 6C). Impurity abundance estimates were performed for batches of ALC-0315 using an LC-UV method. Chromatograms depict the elution peaks of ALC-0315 with and without abundant impurities (FIGS. 11A-11C, 12A-12C. Tables 2-3). MS1 spectra of a contaminated batch revealed strong impurity peaks at 866.9 m/z (FIGS. 11A-11C), however, were not present in all batches of ALC-0315 (FIGS. 12A-12C).

TABLE 2
Peak table of contaminated ALC-0315 sample

| Peak # | Retention Time | Area | Height | Area % |
|---|---|---|---|---|
| 1 | 15.478 | 25473440 | 519440 | 98.907 |
| 2 | 18.776 | 52502 | 12086 | 0.204 |
| 3 | 19.859 | 8761 | 2651 | 0.034 |
| 4 | 20.425 | 22537 | 8042 | 0.088 |
| 5 | 20.828 | 59517 | 3014 | 0.231 |
| 6 | 22.817 | 138142 | 16678 | 0.536 |
| Total | | 25754898 | 561911 | 100.00 |

TABLE 3
Peak table of uncontaminated ALC-0315 sample

| Peak # | Retention Time | Area | Height | Area % |
|---|---|---|---|---|
| 1 | 14.959 | 42904743 | 648135 | 99.910 |
| 2 | 18.203 | 8949 | 79 | 0.021 |
| 3 | 18.646 | 9038 | 2329 | 0.021 |
| 4 | 20.571 | 11060 | 267 | 0.026 |
| 5 | 22.833 | 9434 | 1577 | 0.022 |
| Total | | 42943225 | 652387 | 100.00 |

Although the contaminated mRNA-LNPs had similar physical characteristics as uncontaminated mRNA-LNPs as assessed by batch DLS (Table 4), they display ed greatly compromised stability in serum. Contaminated mRNA-LNPs degraded faster in serum such that their abundance after 90 mins (˜5%) was an order of magnitude less than uncontaminated mRNA-LNPs (˜50%) (FIG. 6D).

TABLE 4
Physical properties of mRNA-LNPs
with and without impurity (N = 3)

| mRNA- | | | |
|---|---|---|---|
| LNPs | Rₕ (nm) | PD | ζ (mV) |
| Without | 41.6 ± 2.2 | 0.156 | −0.65 ± 0.56 |
| impurity | | | |
| With | 46.4 ± 3.8 | 0.064 | −1 ± 0.2 |
| impurity | | | |

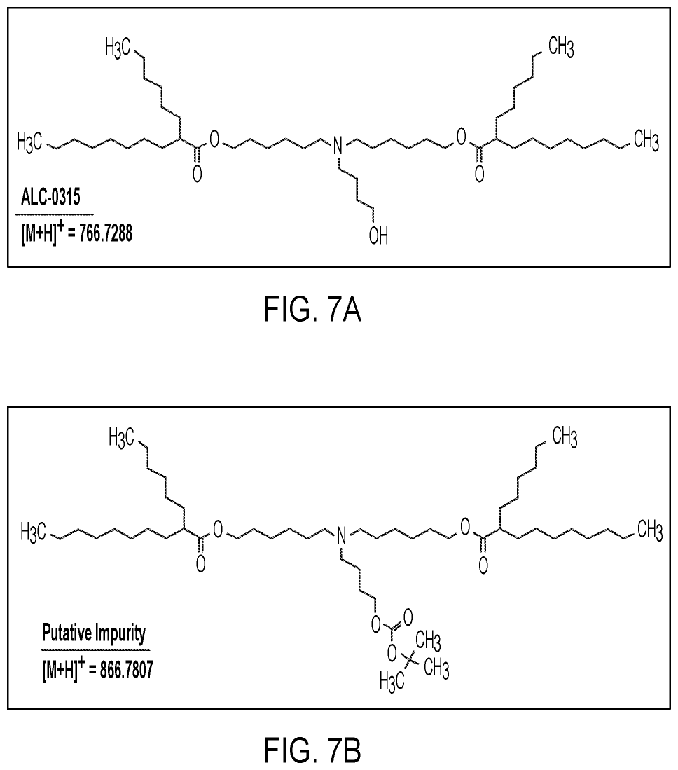

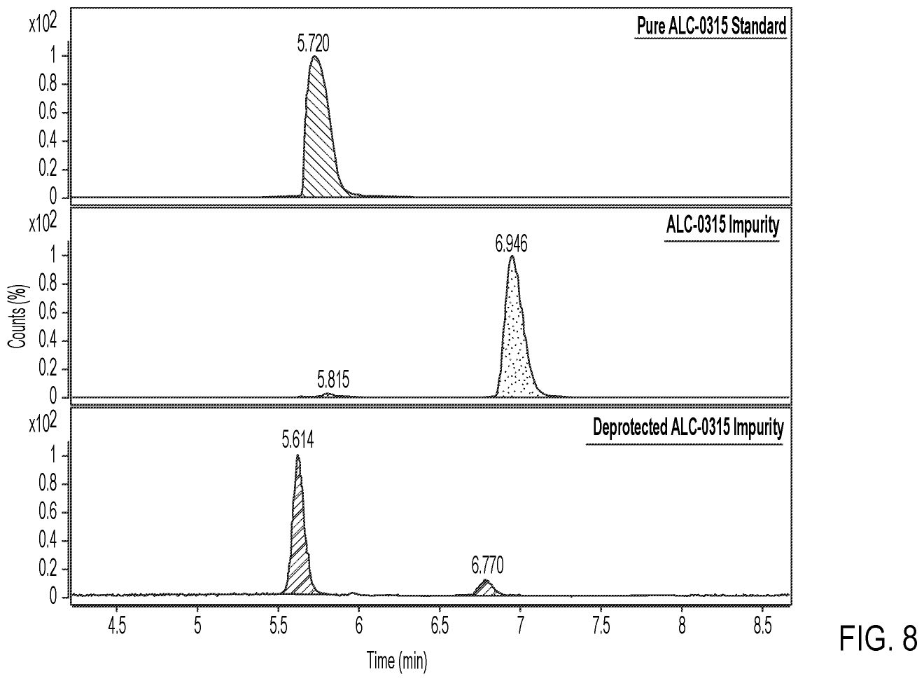

Based on the impurity's monoisotopic mass, contaminated mRNA-LNPs were determined to contain an incompletely deprotected form of ALC-0315 containing an O-Boc group (FIG. 7B). Mildly acidic-labile protection groups have been used in the synthesis of ALC-0315. The impurity peak was subjected to deprotection under acidic conditions before re-analysis by LC-MS. Deprotection successfully converted most of the impurity fraction into ALC-0315, thus confirming its identity (FIG. 8).

### SUMMARY

Although Rₕ measured by batch DLS can be used as an indicator of LNP stability, this can lead to misleading results, particularly when studying LNPs in complex media such as human plasma or serum. High molecular weight plasma or serum components interfere greatly with DLS, thus requiring dilute plasma solutions or LNP recovery techniques such as ultracentrifugation. Unfortunately, dilute solutions do not fully recapitulate the effects of undiluted physiological fluids, and LNP recovery techniques can be prone to bias because they tend to favor the recovery of larger particles. Thus, even significant degradation processes may be overlooked. Moreover, the use of DLS can be problematic because it can only provide relative size profile information and not an accurate number of intact LNPs remaining over time.

The SEC-MALS approach described herein provides a method with improved accuracy to measuring LNP stability in complex media. After separating LNPs from interfering plasma or serum components by SEC, MALS can be used to measure the number of intact LNPs remaining with high sensitivity based on their retention time. LNPs of the Cominarty lipid composition degrade relatively quickly in both plasma and serum (half-life of 80 to 85 min). MALS also revealed a progressive replacement of LNP lipids with plasma components, corroborated by LC-MS/MS. This method also proved useful as a quality control tool as it detected the destabilization of mRNA-LNPs by Boc-protected ALC-0315 impurities. SEC-MALS can be used for lipid formulation optimization and quality control to accelerate therapeutics discovery.

In contrast to measuring nanoparticle degradation using fluorescently labeled nanoparticles, including phospholipid bilayers stabilized by apolipoprotein scaffolds with Alexa Fluor 488, which measured the number of intact LNPs over time after incubation in serum using a fluorescence detector coupled to SEC, the method described here is advantageous, at least in part, because fluorescent labels may influence the stability of nanoparticles, whereas MALS is a highly sensitive label-free technique.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A method of determining the stability of a lipid nanoparticle (LNP) in a subject, comprising:
acquiring a first sample comprising the LNP from the subject;
subjecting the first sample to a size-exclusion chromatography (SEC);
acquiring a multi angle light scattering (MALS) signal from the first sample;
acquiring a second sample comprising the LNP from the subject;
subjecting the second sample to the SEC;
acquiring a second MALS signal from the second sample; and
comparing the first MALS signal and the second MALS signal,
wherein the first and second samples comprise a physiological fluid, and wherein the comparison between the first MALS signal and the second MALS signal is indicative of the stability of the LNP in the subject,
thereby determining the stability of the LNP in the subject.

2. The method of claim 1, wherein the SEC is performed in a dual-column configuration using a first column with a first average pore size and a second column with a second average pore size, and wherein the first average pore size is greater than the second average pore size.

3. The method of claim 2, wherein the first average pore size has a molecular weight cut-off (MWCO) of 1 MDa to 20 MDa, and wherein the second average pore size has an MWCO of 0.1 MDa to 10 MDa.

4. The method of claim 1, wherein the SEC is performed using a polymer-based column having a length of about 100 mm to 1000 mm with a bio-compatible buffer system.

5. The method of claim 1, wherein the MALS is performed at a scattering angle of 15° and 90° and using a laser wavelength of about 500 nm to 800 nm.

6. The method of claim 1, wherein the MALS is performed using a sample cell volume of about 1 μL to 50 μL and a scattering volume of about 0.001 μL to 0.1 μL.

7. The method of claim 1, wherein the MALS is performed at a temperature range of about 20° C. to 70° C. with a temperature stability of no more than ±1° C.

8. The method of claim 1, wherein the MALS is performed at a pH range of about 1-12.

9. The method of claim 1, wherein the size of the LNP is determined.

10. The method of claim 1, wherein the molecular weight (MW) of the LNP is determined.

11. The method of claim 1, wherein the half-life of the LNP is determined.

12. The method of claim 1, wherein the method does not comprise a step of recovering the LNP by ultracentrifugation.

13. The method of claim 1, wherein the method does not comprise a step of diluting the sample to remove a high molecular weight plasma or serum component.

14. The method of claim 1, wherein the method does not comprises a step of labeling the LNP with a fluorophore.

15. The method of claim 1, wherein the LNP is loaded with a nucleic acid.

16. The method of claim 1, wherein the nucleic acid is an mRNA, a small interfering RNA (siRNA), an antisense oligonucleotide (ASO), or a DNA.

17. The method of claim 16, wherein the physiological fluid is plasma, serum, or blood.

18. A system comprising a processor configured to perform the method of claim 1.

19. A method of determining the purity of a lipid nanoparticle (LNP) in a sample, comprising:
acquiring a first aliquot of the sample;
subjecting the first aliquot to a size-exclusion chromatography (SEC);
acquiring a multi angle light scattering (MALS) signal from the first aliquot;
acquiring a second aliquot of the sample;
subjecting the second aliquot to the SEC;
acquiring a second MALS signal from the second aliquot; and
comparing the first MALS signal and the second MALS signal to determine the stability of the LNP in the sample, which is indicative of the purity of the LNP in the sample,
wherein the sample comprises a physiological fluid,
thereby determining the purity of the LNP in the sample.

20. A method of monitoring a process of manufacturing a lipid nanoparticle (LNP), comprising:
acquiring a first sample comprising an LNP from the process of manufacturing the LNP;
subjecting the first sample to a size-exclusion chromatography (SEC); and
acquiring a multi angle light scattering (MALS) signal from the first sample,
acquiring a second sample comprising the LNP from the process of manufacturing the LNP;
subjecting the second sample to the SEC; and
acquiring a second MALS signal from the second sample,
wherein the first and second samples comprise a physiological fluid,
thereby monitoring the process of manufacturing the LNP.
